(19) United States
(12) Patent Application Publication
(10) Pub. No.: US 2007/0275957 A1
(43) Pub. Date: Nov. 29, 2007

(54) USE OF N-DESMETHYLCLOZAPINE TO TREAT HUMAN NEUROPSYCHIATRIC DISEASE

(76) Inventors: David M. Weiner, San Diego, CA (US); Mark R. Brann, Rye, NH (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/671,405

(22) Filed: Feb. 5, 2007

Related U.S. Application Data
(63) Continuation of application No. PCT/US05/27645, filed on Aug. 4, 2005.
Application No. PCT/US05/27645 is a continuation-in-part of application No. 10/913,117, filed on Aug. 5, 2004.
Application No. PCT/US05/27645 is a continuation-in-part of application No. 11/098,892, filed on Apr. 4, 2005.
Application No. PCT/US05/27645 is a continuation-in-part of application No. 11/098,892, filed on Apr. 4, 2005.
Application No. 11/098,892 is a continuation-in-part of application No. 10/913,117, filed on Aug. 5, 2004.
Application No. 10/913,117 is a continuation-in-part of application No. 10/761,787, filed on Jan. 21, 2004.
(60) Provisional application No. 60/617,553, filed on Oct. 8, 2004.
(60) Provisional application No. 60/442,690, filed on Jan. 23, 2003.

(51) Int. Cl. A61K 31/5415

(52) U.S. Cl. 514/225.5

(57) ABSTRACT

Disclosed herein is a method to treat neuropsychiatric diseases including psychosis, affective disorders, dementia, neuropathic pain, and glaucoma. Treatment is carried out by administering a therapeutically effective amount of N-desmethylclozapine to a patient suffering from a neuropsychiatric disease.

### RELATED APPLICATIONS

This application is a continuation of PCT International Application No. PCT/US2005/027645, filed Aug. 4, 2005, which claims the benefit of U.S. application Ser. No. 10/913,117, filed Aug. 5, 2004; U.S. application Ser. No. 11/098,892, filed Apr. 4, 2005; and U.S. Provisional Application No. 60/617,553, filed Oct. 8, 2004, all of which are incorporated herein by reference in their entirety. This application is also a continuation-in-part of U.S. application Ser. No. 11/098,892, filed Apr. 4, 2005; which is a continuation-in-part of U.S. application Ser. No. 10/913,117, filed Aug. 5, 2004; which is a continuation-in-part of U.S. application Ser. No. 10/761,787, filed Jan. 21, 2004; which claims the benefit of U.S. Provisional Application No. 60/442,690, filed Jan. 23, 2003, all of which are incorporated herein by reference in their entirety.

### FIELD OF THE INVENTION

The present invention relates to the discovery of potent muscarinic receptor agonist properties of the dibenzodiazepine compound N-desmethylclozapine, 8-chloro-11-(1-piperazinyl)-5H-dibenzo[b,e][1,4]diazepine, which supports the clinical use of this drug as a superior therapeutic agent for the treatment of pain, glaucoma, dementia, affective disease, and psychosis.

### BACKGROUND OF THE INVENTION

The physiological actions of the hormone/neurotransmitter acetylcholine are mediated, in part, by muscarinic acetylcholine receptors. Muscarinic receptors comprise a family of five (M1-M5) transmembrane proteins that mediate slow, modulatory signalling in cells and tissues expressing these genes. Muscarinic receptors are the targets of a number of therapeutically useful agents (1, 2). Peripherally, muscarinic receptors mediate the actions of acetylcholine in the parasympathetic nervous system. Peripherally acting muscarinic receptor agonists are therapeutically useful in lowering intra-ocular pressure in patients with glaucoma (3). Compounds that potentiate the central actions of acetylcholine as well as centrally acting muscarinic receptor agonists have both demonstrated clinical utility in the treatment of a number of neuropsychiatric diseases (1, 2, 4-7).

The actions of acetylcholine are terminated by degradation of the molecule by acetylcholinesterase enzymes. Inhibition of these enzymes within the central nervous system leads to increased concentrations of acetylcholine at muscarinic receptors. A number of acetylcholinesterase inhibitors have been developed and are in routine clinical use as cognitive enhancing agents in dementia (4).

A number of centrally acting muscarinic agonist have been the subject of clinical testing. One of these, Xanomeline, has been shown to possess efficacy in controlling psychosis and related behavioral disturbances observed in Alzheimer's Disease patients (5). Further, it has recently been demonstrated that xanomeline is efficacious in treating schizophrenia (6). Interestingly, it displayed efficacy against both positive and negative symptoms, and did not induce adverse motoric effects in initial clinical studies in schizophrenics. These data suggest that compounds with muscarinic receptor agonist properties are likely to be efficacious in treating the behavioral disturbances common to neurodegenerative disease such as Alzheimers Disease and as antipsychotics to treat human psychoses, but only if they are tolerated in these patient populations. Additionally, muscarinic receptor agonists have shown activity in pre-clinical models of neuropathic pain states (7).

### SUMMARY OF THE INVENTION

Disclosed herein is a method of treating psychosis comprising: identifying a subject suffering from one or more symptoms of psychosis; and contacting the subject with a therapeutically effective amount of N-desmethylclozapine; whereby the one or more symptoms of psychosis are ameliorated. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In one embodiment, the method further comprises contacting the subject with an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine repuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating affective disorders comprising: identifying a subject suffering from one or more symptoms of an affective disorder; and administering a therapeutically effective amount of N-desmethylclozapine to the subject, whereby the one or more symptoms of the affective disorder are ameliorated. In one embodiment, the subject is human. In one embodiment, the affective disorder is depression. In another embodiment, the affective disorder is mania. In some embodiments, the therapeutically effective amount of N-desmethylclozapine is administered as a single dose. In other embodiments, the therapeutically effective amount of N-desmethylclozapine is administered as a plurality of doses. In one embodiment, the method further comprises administering to the subject an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating dementia, comprising: identifying a subject suffering from one or more symptoms of dementia; and administering a therapeutically effective amount of N-desmethylclozapine to said subject, whereby a desired clinical effect is produced. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In one embodiment, the dementia manifests as a cognitive impairment. In another embodiment, the dementia manifests as a behavioral disturbance. In one embodiment, the method further comprises administering to the subject an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating neuropathic pain comprising: identifying a subject suffering from one or more symptoms of neuropathic pain; and contacting said subject with a therapeutically effective amount of N-desmethylclozapine, whereby the symptoms of neuropathic pain are reduced. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In one embodiment, the method further comprises contacting the subject with an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

Also disclosed herein is a method of treating glaucoma comprising: identifying a subject suffering from one or more symptoms of glaucoma; and contacting said subject with a therapeutically effective amount of N-desmethylclozapine, whereby the symptoms of glaucoma are reduced. In one embodiment, the subject is human. In some embodiments, the therapeutically effective amount of N-desmethylclozapine is administered as a single dose. In other embodiments, the therapeutically effective amount of N-desmethylclozapine is administered as a plurality of doses. In some embodiments, the symptoms of glaucoma are selected from the group consisting of elevated intraocular pressure, optic nerve damage, and decreased field of vision. In one embodiment, the method further comprises contacting the subject with an additional therapeutic agent. In one embodiment, the subject is contacted with the additional therapeutic agent subsequent to the contacting with N-desmethylclozapine. In another embodiment, the subject is contacted with the additional therapeutic agent prior to the contacting with N-desmethylclozapine. In still another embodiment, the subject is contacted with the additional therapeutic agent substantially simultaneously with N-desmethylclozapine. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptics, prostenoids and alpha and beta adrenergic agonists.

Also disclosed herein is a pharmaceutical composition comprising a pharmaceutically effective amount of N-desmethylclozapine and an additional therapeutic agent. In some embodiments, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists. In some embodiments, the additional therapeutic agent is selected from the group consisting of a phenothiazine, phenylbutylpiperadine, debenzapine, benzisoxidil, and salt of lithium. In some embodiments, the additional therapeutic gent is selected from the group consisting of chlorpromazine (Thorazine®), mesoridazine (Serentil®), prochlorperazine (Compazine®), thioridazine (Mellaril®), haloperidol (Haldol®), pimozide (Orap®), clozapine (Clozaril®), loxapine (Loxitane®), olanzapine (Zyprexa®), quetiapine (Seroquel®), risperidone (Risperidal®), ziprasidone (Geodon®), lithium carbonate, Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, Zyprexa, and pharmaceutically acceptable salts thereof In some embodiments the selective serotonin reuptake inhibitor is selected from the group consisting of fluoxetine, fluvoxamine, sertraline, paroxetine, citalopram, escitalopram, sibutramine, duloxetine, venlafaxine, and pharmaceutically acceptable salts and prodrugs thereof. In some embodiments, the norepinephrine reuptake inhibitor is selected from the group consisting of thionisoxetine and reboxetine. In some embodiments, the dual serotonin and norepinephrine reuptake inhibitor is selected from the group consisting of duloxetine, milnacripran and fluvoxamine. In some embodiments, the dopamine agonist is selected from the group consisting of cabergoline, amantadine, lisuride, pergolide, ropinirole, pramipexole, L-DOPA and bromocriptine. In one embodiment, the inverse serotonin agonists selected from the group consisting of N-(1-methylpiperidin-4-yl)-N-(4-flourophenylmethyl)-N′-(4-(2-methylpropyloxy)phenylmethyl)carbamide, MDL 100,907, SR-43694B (eplivanserin), ritanserin, ketanserin, mianserin, cinanserin, mirtazepine, cyproheptadine and cinnarizine.

One embodiment of the present invention includes, a method of treating cognitive impairment comprising identifying a subject in need of improvement of cognition and administering an amount of N-desmethylclozapine to said subject, which is therapeutically effective in improving the cognition of said subject.

In some aspects of this embodiment, the subject is human. In some aspects of this embodiment, the therapeutically effective amount of N-desmethylclozapine is administered as a single dose. In other aspects of this embodiment, the therapeutically effective amount of N-desmethylclozapine is administered as a plurality of doses.

In further aspects of this embodiment, the method further comprises contacting the subject with an additional therapeutic agent. For example, the subject may be contacted with said additional therapeutic agent subsequent to said contacting with N-desmethylclozapine. Alternatively, the subject may be contacted with said additional therapeutic agent prior to said contacting with N-desmethylclozapine.

In some cases, the subject is contacted with said additional therapeutic agent substantially simultaneously with N-desmethylclozapine. In some cases, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists. In some aspects of this embodiment, the subject suffers from a condition selected from the group consisting of hallucinations, delusions, disordered thought, behavioral disturbance, aggression, suicidality, mania, anhedonia, flattening of affect, affective disorders, depression, mania, dementia, neuropathic pain, glaucoma and two or more any of the foregoing conditions.

Another embodiment of the present invention includes method of ameliorating at least one symptom of a condition where it is beneficial to increase the level of activity of an M1 muscarinic receptor comprising determining that a subject would benefit from an increased level of activity of an M1 muscarinic receptor and administering an amount of N-desmethylclozapine which is therapeutically effective to increase the level of activity of the M1 muscarinic receptor and to ameliorate said at least one symptom to the subject. In some aspects of this embodiment, the therapeutically effective amount of N-desmethylclozapine is administered as a single dose. In other aspects of this embodiment, the therapeutically effective amount of N-desmethylclozapine is administered as a plurality of doses. In further aspects of this embodiment, the method further comprises contacting the subject with an additional therapeutic agent. For example, the subject may be contacted with said additional therapeutic agent subsequent to said contacting with N-desmethylclozapine. Alternatively, the subject may be contacted with said additional therapeutic agent prior to said contacting with N-desmethylclozapine. In some cases, the subject is contacted with said additional therapeutic agent substantially simultaneously with N-desmethylclozapine. In some cases, the additional therapeutic agent is selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists. In some aspects of this embodiment, the subject suffers from a condition selected from the group consisting of hallucinations, delusions, disordered thought, behavioral disturbance, aggression, suicidality, mania, anhedonia, flattening of affect, affective disorders, depression, mania, dementia, neuropathic pain, glaucoma and two or more any of the foregoing conditions.

Another aspect of the present invention is a method for ameliorating one or more symptoms of psychosis, comprising administering to a subject exhibiting one or more symptoms of psychosis a therapeutically effective amount of N-desmethylclozapine essentially free of clozapine. One embodiment further comprises identifying a subject exhibiting one or more symptoms of psychosis. In one embodiment, the psychosis is induced by exposure of the subject to one or more medications. In one embodiment, the subject is human. In one embodiment, the N-desmethylclozapine is administered as a single daily dose or administered in divided doses. In one embodiment, the N-desmethylclozapine is administered two, three or four times daily.

Another aspect of the present invention is a method of ameliorating one or more symptoms of an affective disorder, comprising administering to a subject exhibiting one or more symptoms of an affective disorder a therapeutically effective amount of N-desmethylclozapine essentially free of clozapine. One embodiment further comprises identifying a subject exhibiting one or more symptoms of an affective disorder. In one embodiment, the affective disorder is depression. In one embodiment, the affective disorder is mania.

Another aspect of the present invention is a method of ameliorating one or more symptoms of dementia, comprising administering to a subject exhibiting one or more symptoms of dementia a therapeutically effective amount of N-desmethylclozapine essentially free of clozapine. One embodiment further comprises identifying a subject exhibiting one or more symptoms of dementia. In one embodiment, the dementia comprises cognitive impairment. In one embodiment, the dementia comprises behavioral disturbances.

Another aspect of the present invention is a method of ameliorating one or more symptoms of neuropathic pain, comprising administering to a subject exhibiting one or more symptoms of neuropathic pain a therapeutically effective amount of N-desmethylclozapine essentially free of clozapine. One embodiment further comprises identifying a subject exhibiting one or more symptoms of neuropathic pain.

Another aspect of the present invention is a method of ameliorating one or more symptoms of glaucoma, comprising administering to a subject exhibiting one or more symptoms of glaucoma a therapeutically effective amount of N-desmethylclozapine essentially free of clozapine. One embodiment further comprises identifying a subject exhibiting one or more symptoms of glaucoma.

Another aspect of the present invention is a method of ameliorating one or more symptoms of psychosis, comprising administering to a subject N-desmethylclozapine in combination with another anti-psychotic agent, wherein at least a portion of the N-desmethylclozapine is administered by directly introducing N-desmethylclozapine to the subject. In one embodiment, directly introducing N-desmethylclozapine to the subject comprises orally administering N-desmethylclozapine. In one embodiment, directly introducing N-desmethylclozapine to the subject comprises intravenous injection of N-desmethylclozapine. In one embodiment, the other anti-psychotic agent is selected from the group consisting of a phenothiazine, phenylbutylpiperadine, debenzapine, benzisoxidil, and a salt of lithium. In one embodiment, the phenothiazine is selected from the group consisting of chlorpromazine (Thorazine®), mesoridazine (Serentil®), prochlorperazine (Compazine®), and thioridazine (Mellaril®). In one embodiment, the phenylbutylpiperadine is selected from the group consisting of haloperidol (Haldol®) and pimozide (Orap®). In one embodiment, the debenzapine is selected from the group consisting of clozapine (Clozaril®), loxapine (Loxitane®), olanzapine (Zyprexa®) and quetiapine (Seroquel®). In one embodiment, the benzisoxidil is selected from the group consisting of resperidone (Resperidal®) and ziprasidone (Geodon®). In one embodiment, the salt of lithium is lithium carbonate. In one embodiment, the antipsychotic agent is selected from the group consisting of Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, and Zyprexa, or pharmaceutically acceptable salts thereof.

Another aspect of the present invention is a method of ameliorating one or more symptoms of psychosis, including administering to a subject exhibiting one or more symptoms of psychosis a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at muscarinic receptors.

Another aspect of the present invention is a method of ameliorating one or more symptoms of an affective disorder, including administering to a subject exhibiting one or more symptoms of an affective disorder a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at muscarinic receptors.

Another aspect of the present invention is a method of ameliorating one or more symptoms of dementia, including administering to a subject exhibiting one or more symptoms of dementia a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at muscarinic receptors.

Another aspect of the present invention is a method of ameliorating one or more symptoms of neuropathic pain, including administering to a subject exhibiting one or more symptoms of neuropathic pain a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at muscarinic receptors.

Another aspect of the present invention is a method of ameliorating one or more symptoms of glaucoma, including administering to a subject exhibiting one or more symptoms of glaucoma a therapeutically effective amount of a pharmaceutical composition comprising N-desmethylclozapine and a pharmaceutically acceptable excipient or diluent, wherein the amount of any clozapine administered is low enough such that the combined N-desmethylclozapine and clozapine result in a net agonism at muscarinic receptors.

### BRIEF DESCRIPTION OF THE DRAWINGS

FIG. 1 is a graph showing the results of agonist activity of N-desmethylclozapine at M1 muscarinic acetylcholine receptors in R-SAT Assays.

FIG. 2 is a graph showing the results of agonist activity of N-desmethylclozapine at M1 musacrinic acetylcholine receptors in Phosphatidyl Inositol Assay.

FIG. 3 shows photographs of MAP kinase activation in rat hippocampus following parenteral administration of N-desmethylclozapine.

FIG. 4A shows a graph of the muscarinic M1 receptor agonist activity of a library of 462 compounds as determined by R-SAT assays. M1 receptor efficacy data shown are derived from the 1-micromolar concentration of compound, and are reported as percentage efficacy relative to the maximal response observed for a saturating 40-micromolar concentration of carbachol (100%). FIGS. 4B-D shows a graph of PI hydrolysis data utilizing Chinese Hamster Ovary cells stably transfected with the human M1 receptor gene. Panel B depicts agonist responses reported as the percentage response observed for carbachol. Drugs depicted are carbachol (squares), clozapine (triangles), and N-desmethylclozapine (circles), with observed potencies (pEC₅₀) of: carbachol (5.7), N-desmethylclozapine (6.7), and clozapine (no response). Panel C depicts competitive antagonist responses obtained in the presence of a 3-micromolar concentration of carbachol, and are reported as the percentage response observed for atropine (100%). Drugs depicted are atropine (squares), clozapine (triangles), and N-desmethylclozapine (circles), with observed potencies (pKi) of: atropine (8.5), N-desmethylclozapine (no response), and clozapine (7.1). Panel D depicts competitive antagonist responses obtained in the presence of a 0.15-micromolar concentration of N-desmethylclozapine, and are reported as the percentage response observed for atropine (100%). Drugs depicted are atropine (squares), and clozapine (triangles), with observed potencies (pKi) of: atropine (8.4), and clozapine (7.6).

FIG. 5 shows M1 muscarinic receptor agonist activity of N-desmethylclozapine in mouse hippocampus. Phospho-MAPK immunoreactivity in the cell bodies and proximal dendrites of CA1 pyramidal cells (highlighted by arrows) is shown following the administration of vehicle (A), clozapine at 30 mg/kg (B), N-desmethylclozapine at 10 (C), 30 (D), 100 (E), or N-desmethylclozapine (30 mg/kg) and scopolamine (0.3 mg/kg, i.p.)(F).

FIG. 6 shows the quantification of M1 muscarinic receptor agonist activity of N-desmethylclozapine in mouse hippocampus. Quantification of phospho-MAPK immunoreactivity was performed via computer calculated optical density measurements of the CA1 region of the hippocampus from four mice, where (*) indicates a significant difference to vehicle treatment using a one factor ANOVA post-hoc Dunnett's test (F _{(5,23)}=10.88: P<0.0001).

FIG. 7 shows the results of an R-SAT assay with a combination of 150 nM NDMC and varying concentrations of clozapine.

FIG. 8 shows the results of a PI hydrolysis assay with a combination of 150 nM NDMC and varying concentrations of clozapine.

### DETAILED DESCRIPTION OF THE PREFERRED EMBODIMENT

Definitions

N-desmethylclozapine, 8-chloro-11-(1-piperazinyl)-5H-dibenzo[b,e][1,4]diazepine, also known as NDMC, is defined as the compound having the molecular structure depicted in Formula (I).

An “agonist” is defined as a compound that increases the basal activity of a receptor (i.e. signal transduction mediated by the receptor).

An “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity.

A partial agonist is defined as an agonist that displays limited, or less than complete, activity such that it fails to activate a receptor in vitro, functioning as an antagonist in vivo.

The term “subject” refers to an animal, preferably a mammal, and most preferably a human, who is the object of treatment, observation or experiment.

The term “therapeutically effective amount” is used to indicate an amount of an active compound, or pharmaceutical agent, that elicits the biological or medicinal response indicated. This response may occur in a tissue, system, animal or human that is being sought by a researcher, veterinarian, medical doctor or other clinician, and includes alleviation of the symptoms of the disease being treated.

In certain embodiments, the method disclosed herein includes administering a therapeutically effective amount of NDMC to a subject for the purpose of treating psychosis.

In certain embodiments, the above method for treating psychosis comprises identifying a subject suffering from one or more symptoms of psychosis; and contacting the subject with a therapeutically effective amount of N-desmethylclozapine; whereby the one or more symptoms of psychosis are ameliorated.

In some embodiments, the symptom is cognitive impairment associated with psychosis. In other embodiments, the subject suffering from psychosis exhibits more than one symptom of psychosis. In certain embodiments, one of the symptoms is cognitive impairment while another symptoms is one or more of hallucinations, delusions, disordered thought, behavioral disturbance, aggression, suicidality, mania, anhedonia, or flattening of affect.

In a further embodiment, the method includes administering a therapeutically effective amount of NDMC to a subject for the purpose of treating depression or mania.

In a still further embodiment, the method includes administering a therapeutically effective amount of NDMC to a subject for the purpose of treating the psychiatric and other behavioral disturbances characteristic of dementia or cognitive impairment of any origin.

In a still further embodiment, the method includes administering a therapeutically effective amount of NDMC to a subject for the purpose of treating neuropathic pain.

The present inventors have profiled a large series of drugs that have utility in treating human disease for functional activity at the five human muscarinic receptor subtypes. With the exception of known muscarinic drugs, only two agents studied (out of more than 500) displayed muscarinic receptor agonist activity. One was the atypical antipsychotic clozapine (8). In vitro, this compound has been shown to possess weak partial agonist/antagonist activity at muscarinic M1, M2, and M4 receptors (9, 10), while in vivo it is generally considered to display muscarinic receptor antagonist properties. The other was the related compound N-desmethylclozapine.

Administration of clozapine to human subjects results in the formation of two major metabolites N-desmethylclozapine (NDMC) and clozapine-N-oxide (11). However, clozapine-N-oxide is a polar metabolite that is rapidly excreted and likely does not contribute to the biological activity of the parent compound. A correlation exists between the dose of clozapine administered to a subject, and the serum levels of total clozapine moieties, yet the levels of NDMC can vary widely between individual subjects (12). Generally, NDMC constitutes 40-75% of the total serum clozapine concentrations during steady state kinetics in humans (13). Conflicting data exists as to the ability of NDMC to penetrate the blood brain barrier and impart centrally mediated activity (14, 15). These observations demonstrate that NDMC has been routinely administered to human subjects, and is well tolerated. Few data exist as to the molecular properties of NDMC. NDMC has been shown to possess antagonist activity at 5HT_{2C} and D2 receptors (16), but no data on its interaction with muscarinic receptors has been reported.

Surprisingly, and unlike the closely related compound clozapine, it has been found that the compound N-desmethylclozapine (NDMC) possesses heretofore unappreciated functional activity as a muscarinic receptor agonist. Ex vivo experiments have demonstrated that NDMC crosses the blood brain barrier and acts as an agonist at central muscarinic receptors in rats. These observations have practical applications that support the use of NDMC as an antipsychotic, antimania agent, antidementia agent, and as a therepeutic agent to treat glaucoma or neuropathic pain. Thus, in one aspect, disclosed herein is a method of agonizing the activity of a muscarinic receptor comprising contacting the receptor with an effective amount of NDMC. In another aspect, disclosed herein is a method of treating a subject suffering from a muscarinic receptor related disorder comprising indentifying a subject in need thereof and administering to the subject a therapeutically effective amount of NDMC.

By “muscarinic related disorder,” it is meant a disorder whose symptoms are ameliorated by agonizing a muscarinic receptor.

In another aspect, disclosed herein is a method of ameliorating one or more symptoms associated with schizophrenia or psychosis of any origin in a subject, comprising administering to the subject a therapeutically effective amount of NDMC. In some embodiments, the method comprises contacting a subject with a pharmacologically active dose of NDMC, for the purpose of controlling the positive (hallucinations and delusion) and negative (apathy, social withdrawal, anhedonia) symptoms of schizophrenia or related psychosis. In one embodiment, the NDMC administered to ameliorate one or more symptoms associated with schizophrenia or psychosis is essentially free of clozapine. By “essentially free of clozapine,” it is meant that no appreciable amount of clozapine may be detected in the blood stream of the subject at the same time that NDMC is detectable in the blood stream of the subject. In one embodiment, the amount of any clozapine administered with the NDMC is low enough such that the combined NDMC and clozapine administered result in a net agonism at muscarinic receptors. In one embodiment, some amount of clozapine is administered but it is low enough such that the combined NDMC and clozapine administered result in a net agonism at muscarinic receptors. In one embodiment, the ratio of NDMC to clozapine is high enough to have a beneficial effect due to net agonism at muscarinic receptors. In various embodiments, the ratio of NDMC to clozapine is at least about 100:1, 50:1, 10:1, 9:1, 7:1, 5:1, or 3:1.

In another aspect, disclosed herein is a method of ameliorating one or more symptoms associated with affective disorders, including major depression, mania, bipolar disorder, and suicide, in a subject, comprising administering to the subject a therapeutically effective amount of NDMC. In some embodiments, the method comprises contacting a subject with a pharmacologically active dose of NDMC, for the purpose of controlling the symptoms observed during major depression or manic depression. In one embodiment, the NDMC administered to ameliorate one or more symptoms associated with affective disorders is essentially free of clozapine. In one embodiment, the amount of any clozapine administered with the NDMC is low enough such that the combined NDMC and clozapine administered result in a net agonism at muscarinic receptors. In one embodiment, some amount of clozapine is administered but it is low enough such that the combined NDMC and clozapine administered result in a net agonism at muscarinic receptors.

In another aspect, disclosed herein is a method of ameliorating one or more symptoms associated with Alzheimer's Disease and related neurodegenerative disorders in a subject, comprising administering to the subject a therapeutically effective amount of NDMC. In some embodiments, the method comprises contacting a subject with a pharmacologically active dose of NDMC, for the purpose of improving the cognitive deficits, and controlling the associated behavioral abnormalities, observed in degenerative dementias. In one embodiment, the NDMC administered to ameliorate one or more symptoms associated with dementia is essentially free of clozapine. In one embodiment, the amount of any clozapine administered with the NDMC is low enough such that the combined NDMC and clozapine administered result in a net agonism at muscarinic receptors. In one embodiment, some amount of clozapine is administered but it is low enough such that the combined NDMC and clozapine administered result in a net agonism at muscarinic receptors.

In another aspect, disclosed herein is a method of ameliorate one or more symptoms associated with neuropathic pain in a subject, comprising identifying a subject in need thereof and administering to the subject a therapeutically effective amount of NDMC. In some embodiments, the method comprises contacting a subject with a pharmacologically active dose of NDMC, for the purpose of controlling the dysthesthetic, hyperalgesic, and other altered nociceptive symptoms observed in neuropathic pain states regardless of their etiology. In one embodiment, the NDMC administered to ameliorate one or more symptoms associated with neuropathic pain is essentially free of clozapine. In one embodiment, the amount of any clozapine administered with the NDMC is low enough such that the combined NDMC and clozapine administered result in a net agonism at muscarinic receptors. In one embodiment, some amount of clozapine is administered but it is low enough such that the combined NDMC and clozapine administered result in a net agonism at muscarinic receptors.

In another aspect, disclosed herein is a method of ameliorating one or more symptoms associated with glaucoma in a subject, comprising administering to the subject a therapeutically effective amount of NDMC. In some embodiments, the method comprises contacting a subject with a pharmacologically active dose of NDMC, for the purpose of controlling the raised intra-ocular pressure observed in glaucoma, regardless of its etiology. In one embodiment, the NDMC administered to ameliorate one or more symptoms associated with glaucoma is essentially free of clozapine. In one embodiment, the amount of any clozapine administered with the NDMC is low enough such that the combined NDMC and clozapine administered result in a net agonism at muscarinic receptors. In one embodiment, some amount of clozapine is administered but it is low enough such that the combined NDMC and clozapine administered result in a net agonism at muscarinic receptors.

Surprisingly, NDMC possesses potent agonist activity at the human muscarinic receptors. It is further disclosed herein that NDMC can cross the blood brain barrier, and function in vivo as a muscarinic receptor agonist measured via the activation of MAP kinase activity in rat hippocampus. The molecular activities of NDMC, as identified by the present methods, combined with the known clinical efficacy of compounds that possess a similar molecular pharmacological profile, indicate that NDMC can be used to alleviate or treat disorders or conditions associated with human psychosis, affective disease, degenerative dementia, glaucoma, and neuropathic pain.

In another aspect, disclosed herein is a method of activating an M1 muscarinic receptor comprising contacting the receptor with N-desmethylclozapine.

In a further aspect, disclosed herein is a method of ameliorating at least one symptom of a condition where it is beneficial to increase the level of activity of an M1 muscarinic receptor comprising administering N-desmethylclozapine to a subject in need thereof.

Preparation of N-desmethylclozapine (NDMC)

N-desmethylclozapine (NDMC) has the structure of Formula (I).

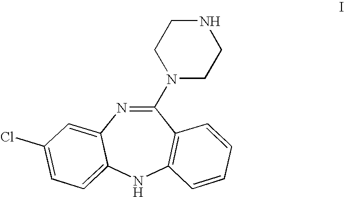

NDMC is prepared as previously described (17). The dibenzo-diazepine-lactam precursor (II) is converted to the thiolactam (III) using phosphorus pentasulfide, followed by alkylation with e.g. dimethyl sulfate to give the imino thioether (IV). Aminolysis of the thioether with an excess of piperazine gives the desired N-desmethylclozapine (I). Alternatively, the dibenzo-diazepine-lactam (II) may be converted into the imino-chloride (V) by treatment with a halogenating agent such as phosphorus pentachloride and the product V is converted to N-desmethylclozapine (I) by reaction with piperazine.

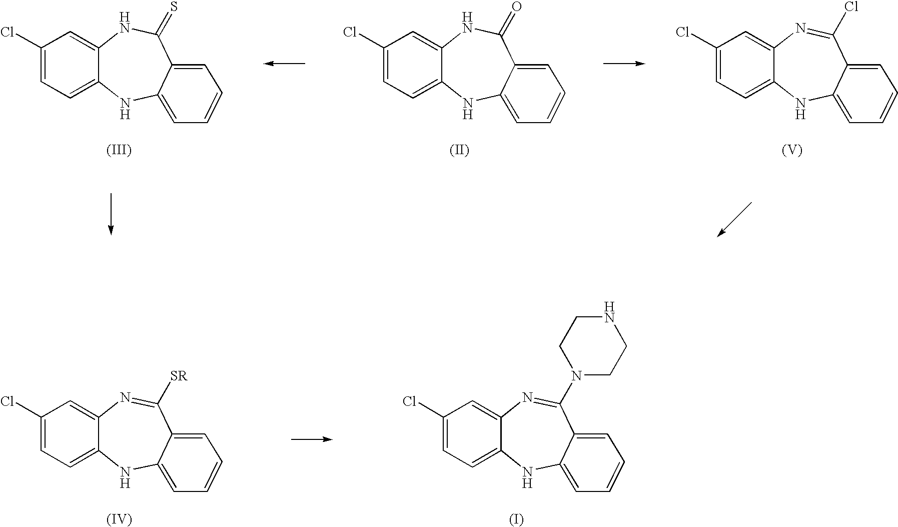

NDMC may be formulated in pharmaceutical compositions comprising NDMC together with a pharmaceutically acceptable dilutant or excipient. Such compositions may be formulated in an appropriate manner and in accordance with accepted practices such as those disclosed in Remington's Pharmaceutical Sciences, Gennaro, Ed., Mack Publishing Co., Easton Pa., 1990. In some embodiments, a pharmaceutical composition comprising NDMC is provided that is essentially free of clozapine.

Advantageously, NDMC may be administered in a single daily dose, or the total daily dosage may be administered as a plurality of doses, (e.g. divided doses two, three or four times daily). Furthermore, compound for the present invention may be administered in intranasal form via topical use of suitable intranasal vehicles, or via transdermal routes, or via topical use of ocular formulations, or using those forms of transdermal skin patches well known to persons skilled in the art.

The dosage regimen of NDMC can be selected in accordance with a variety of factors. These include type, species, age, weight, sex and medical condition of the patient; the severity of the condition to be treated; the route of administration; the renal and hepatic function of the patient; and the particular compound employed. A physician of ordinary skill can readily determine and prescribe the effective amount of the drug required to prevent, counter or arrest the progress of the disease or disorder that is being treated.

The daily dosage of the products may be varied over a wide range from 0.01 to 1000 mg per adult human per day. An effective amount of the drug is ordinarily supplied at a dosage level of about 0.0001 mg/kg to about 25 mg/kg body weight per day. Preferably, the range is from about 0.001 to 10 mg/kg of body weight per day, and especially from about 0.001 mg/kg to 1 mg/kg of body weight per day. The compounds may be administered on a regimen of 1 to 4 times per day.

NDMC may be used alone at appropriate dosages defined by routine testing in order to obtain optimal pharmacological effect, while minimizing any potential toxic or otherwise unwanted effects. In addition, it is believed that NDMC may be used as adjunctive therapy with known drugs to reduce the dosage required of these traditional drugs, and thereby reduce their side effects.

In some embodiments, NDMC is administered in combination with one or more additional therapeutic agents. The additional therapeutic agents can include, but are not limited to, a neuropsychiatric agent. As used herein, a “neuropsychiatric agent” refers to a compound, or a combination of compounds, that affects the neurons in the brain either directly or indirectly, or affects the signal transmitted to the neurons in the brain. Neuropsychiatric agents, therefore, may affect a person's psyche, such as the person's mood, perception, nociception, cognition, alertness, memory, etc. In certain embodiments, the neuropsychiatric agent may be selected from the group consisting of monoamine reuptake inhibitors, selective serotonin reuptake inhibitors, norepinephrine reuptake inhibitors, dual serotonin and norepinephrine reuptake inhibitors, dopamine agonists, antipsychotic agents, inverse serotonin agonists, serotonin antagonists, serotonin 2 inverse agonists, serotonin 2 antagonists, serotonin1A agonists, antiepileptic and peripherally acting muscarinic antagonists.

In some embodiments, the antipsychotic agent may be selected from the group consisting of a phenothiazine, phenylbutylpiperadine, debenzapine, benzisoxidil, and salt of lithium. The phenothiazine group of compounds may be selected from the group consisting of chlorpromazine (Thorazine®), mesoridazine (Serentil®), prochlorperazine (Compazine®), and thioridazine (Mellaril®). The phenylbutylpiperadine group of compounds may be selected from the group consisting of haloperidol (Haldol®), and pimozide (Orap®). The debenzapine group of compounds may be selected from the group consisting of clozapine (Clozaril®), loxapine (Loxitane®), olanzapine (Zyprexa®) and quetiapine (Seroquel®). The benzisoxidil group of compounds may be selected from the group consisting of resperidone (Resperidal®) and ziprasidone (Geodon®). The salt of lithium may be lithium carbonate. In some embodiments, the antipsychotic agent may be selected from the group consisting of Aripiprazole (Abilify), Clozapine, Clozaril, Compazine, Etrafon, Geodon, Haldol, Inapsine, Loxitane, Mellaril, Moban, Navane, Olanzapine (Zyprexa), Orap, Permitil, Prolixin, Phenergan, Quetiapine (Seroquel), Reglan, Risperdal, Serentil, Seroquel, Stelazine, Taractan, Thorazine, Triavil, Trilafon, and Zyprexa, or pharmaceutically acceptable salts thereof.

In certain embodiments, the selective serotonin reuptake inhibitor is selected from the group consisting of fluoxetine, fluvoxamine, sertraline, paroxetine, citalopram, escitalopram, sibutramine, duloxetine, and venlafaxine, and pharmaceutically acceptable salts or prodrugs thereof.

In other embodiments, the norepinephrine reuptake inhibitor is selected from the group consisting of thionisoxetine and reboxetine.

In further embodiments, the dopamine agonist is selected from the group consisting of cabergoline, amantadine, lisuride, pergolide, ropinirole, pramipexole, and bromocriptine.

In another embodiment, the inverse serotonin 2A agonist is N-(1-methylpiperidin-4-yl)-N-(4-flourophenylmethyl)-N′-(4-(2-methylpropyloxy)phenylmethyl)carbamide, MDL 100,907, SR-43694B (eplivanserin), rtianserin, ketanserin, mianserin, cinanserin, mirtazepine, cyproheptadine and cinnarizine.

In another aspect, the present disclosure is directed to a method of treating neuropsychiatric disorder in a patient comprising identifying a patient in need thereof and administering to said patient a therapeutically effective amount of a pharmaceutical composition comprising a compound of Formula (I) and a neuropsychiatric agent. In yet another aspect, the present disclosure is directed to a method of treating neuropsychiatric disorder in a patient comprising identifying a patient in need thereof and administering to said patient a therapeutically effective amount of a compound of Formula (I) and a therapeutically effective amount of a neuropsychiatric agent.

In some embodiments, NDMC and additional therapeutic agent(s) are administered nearly simultaneously. These embodiments include those in which the compounds are in the same administrable composition, i.e., a single tablet, pill, or capsule, or a single solution for intravenous injection, or a single drinkable solution, or a single dragee formulation or patch, contains the compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments, one of NDMC and an additional therapeutic compound is administered first and then the other one of NDMC and the additional therapeutic compound is administered second. In these embodiments, the patient may be administered a composition comprising one of the compounds and then at some time, a few minutes or a few hours later, be administered another composition comprising the other one of the compounds. Also included in these embodiments are those in which the patient is administered a composition comprising one of the compounds on a routine or continuous basis while receiving a composition comprising the other compound occasionally.

In some embodiments of combination administration, NDMC is administered in combination with another therapeutic agent, wherein at least a portion of the NDMC is administered by directly introducing NDMC to a subject. Thus, for example, clozapine may be administered in combination with NDMC wherein both clozapine and NDMC are directly administered to a subject. A portion of the NDMC administered to the patient will be due to metabolism of clozapine. However, another portion of NDMC will be due to direct administration of NDMC. In one embodiment, directly introducing NDMC to a subject may be accomplished by the subject orally ingesting NDMC. In one embodiment, directly introducing NDMC to a subject may be accomplished by intravenously injecting NDMC into the subject.

Defining the functional pharmacological activity of NDMC at a given receptor can be achieved by a variety of methodologies. A currently favored assay is the Receptor Selection and Amplification Technology (R-SAT) assay disclosed in U.S. Pat. No. 5,707,798, the content of which is hereby incorporated by reference in its entirety.

Defining the functional pharmacological activity of NDMC at a given receptor can be achieved by a variety of methodologies. Another currently favored assay is the PI Hydrolysis assay (18).

Defining the ability of NDMC to penetrate the blood brain barrier and elicit a meaningful biological response can be achieved by a variety of methodologies. A currently favored assay is the hippocampal MAP kinase activation assay (19).

The present invention is further disclosed in the following examples, which are not in any way intended to limit the scope of the invention as claimed.

### EXAMPLES

### Example 1

### Receptor Selection and Amplification Technology

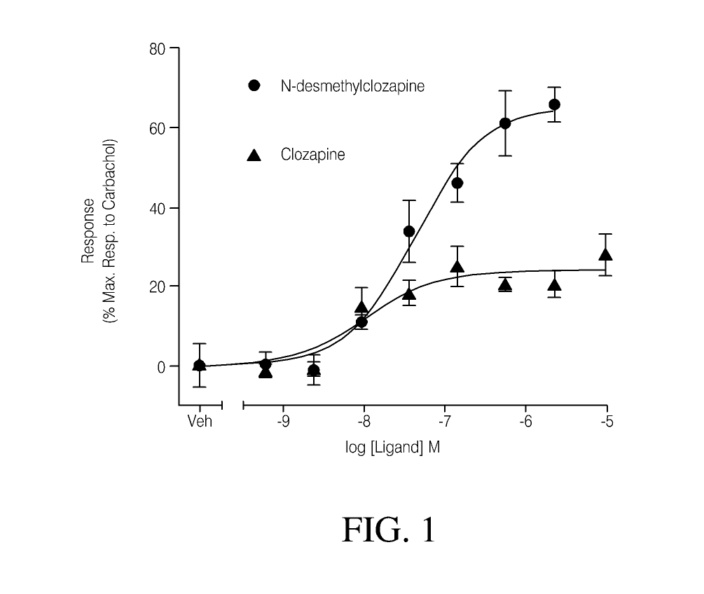

The functional receptor assay, Receptor Selection and Amplification Technology (R-SAT), was used (essentially as disclosed in U.S. Pat. No. 5,707,798, incorporated by reference herein in its entirety) to investigate the functional pharmacological properties of known drugs, including many of their metabolites. These experiments have provided a molecular profile, or fingerprint, for each of these agents. Of all of the agents tested, only one, NDMC, displayed potent M1 acetylcholine receptor agonist activity. FIG. 1 shows the concentration response relationship of clozapine (filled triangles) and N-desmethylclozapine (filled circles) to activate human M1 muscarinic receptors. Data was derived from R-SAT assays as previously previously described (20). Data is plotted as the percentage activation relative to the full muscarinic receptor agonist carbachol versus drug concentration. Veh denotes vehicle.

As shown in FIG. 1, clozapine displays high potency (pEC₅₀ of 7.2) yet limited intrinsic efficacy (<25% relative efficacy) at human M1 receptors. Clozapine is thus defined as a weak partial agonist. Partial agonists lack sufficient intrinsic agonist activity to stimulate the receptor in a manner similar to full agonists. They thus behave as antagonists in vivo. In contrast, NDMC also displays high potency (pEC₅₀ of 7.2) at human M1 receptors, yet it displays significantly greater intrinsic agonist activity at M1 receptors (65% relative efficacy to carbachol), behaving as a robust agonist in R-SAT assays. This increased efficacy suggests that NDMC will act as an agonist in vivo a functional profile distinct from that observed for clozapine.

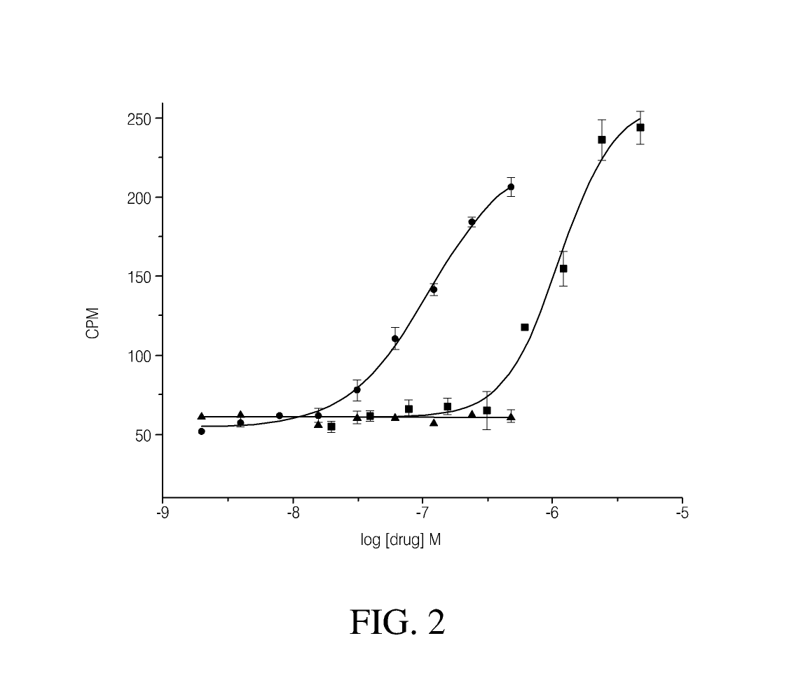

To confirm the observation that NDMC displays increased agonist efficacy at M1 receptors, a PI hydrolysis assay was performed, the results of which are disclosed in FIG. 2 and Table 1. The data in FIG. 2 is derived from PI assays as described in (18). In FIG. 2, the concentration response relationship of carbachol (filled squares), clozapine (filled triangles), and N-desmethylclozapine (filled circles) to activate human M1 muscarinic receptors is shown. Data are plotted as a radioactivity measured in counts per minute versus drug concentration.

TABLE 1

| | M₁ | | |
|---|---|---|---|
| Compound | % Efficacy | pEC50 | n |
| Carbachol | 100% | 6.04 ± 0.05 | 5 |
| Clozapine | No Activity | | |
| N-desmethylclozapine | 65 ± 10 | 7.01 ± 0.06 | 5 |

In Table 1, potency is reported as pEC₅₀ values and efficacy is reported as that relative to the full agonist carbachol, both ± standard deviation. “n” denotes number of experimental determinations. NDMC displays high potency as an M1 agonist in this system (pEC₅₀=7.0), with full efficacy (>65% relative efficacy to carbachol). Thus, two distinct functional assays confirm that NDMC possesses previously unappreciated potent and fully efficacious agonist activity at human M1 muscarinic acetylcholine receptors. This significantly greater positive intrinsic activity of NDMC suggests that it behaves as an M1 receptor agonist in vivo.

Clozapine and NDMC were tested at the remaining muscarinic receptor subtypes. These data are disclosed in Table 2. The data in Table 2 are derived from R-SAT assays as previously described (20). Potency is reported as pEC₅₀ values and efficacy is reported as that relative to the full agonist carbachol, both ± standard deviation. N denotes number of experimental determinations.

TABLE 2

| | M1 | | M2 | | M3 | |
|---|---|---|---|---|---|---|
| Compound | Efficacy | pEC₅₀ | Efficacy | pEC50 | Efficacy | pEC50 |
| Clozapine | 24 ± 3 | 7.63 ± 0.37 | 65 ± 8 | 6.23 ± 0.14 | No response | |
| N-desmethylclozapine | 72 ± 5 | 7.26 ± 0.07 | 106 ± 19 | 6.47 ± 0.21 | 27 ± 4 | 6.49 ± 0.18 |
| Olanzapine | No response | | No response | | No response | |
| N-desmethylolanzapine | No response | | No response | | No response | |
| Xanomeline | 121 ± 6 | 7.20 ± 0.08 | 106 ± 9 | 6.30 ± 0.23 | 66 ± 6 | 6.63 ± 0.21 |
| Carbachol | 101 ± 2 | 6.11 ± 0.03 | 101 ± 5 | 6.23 ± 0.09 | 102 ± 3 | 6.53 ± 0.04 |

| | |
|---|---|
| M4 | M5 |

| | | | | |
|---|---|---|---|---|
| Compound | Efficacy | pEC₅₀ | Efficacy | pEC50 |

| | | | |
|---|---|---|---|
| Clozapine | 57 ± 5 | 7.35 ± 0.10 | No response |

| | | | | |
|---|---|---|---|---|
| N-desmethylclozapine | 87 ± 8 | 6.87 ± 0.17 | 48 ± 6 | 7.63 ± 0.25 |

| | | |
|---|---|---|
| Olanzapine | No response | No response |
| N-desmethylolanzapine | No response | No response |

| | | | | |
|---|---|---|---|---|
| Xanomeline | 116 ± 9 | 7.46 ± 0.14 | 86 ± 12 | 6.59 ± 0.22 |
| Carbachol | 96 ± 3 | 6.53 ± 0.05 | 105 ± 3 | 6.76 ± 0.12 |

NDMC displays increased intrinsic activity at all five muscarinic receptor subtypes when compared to clozapine. The profile of NDMC at human muscarinic receptors is most similar to that observed for the investigational agent Xanomeline, with one important distinction, a significantly lower efficacy at human m3 receptors.

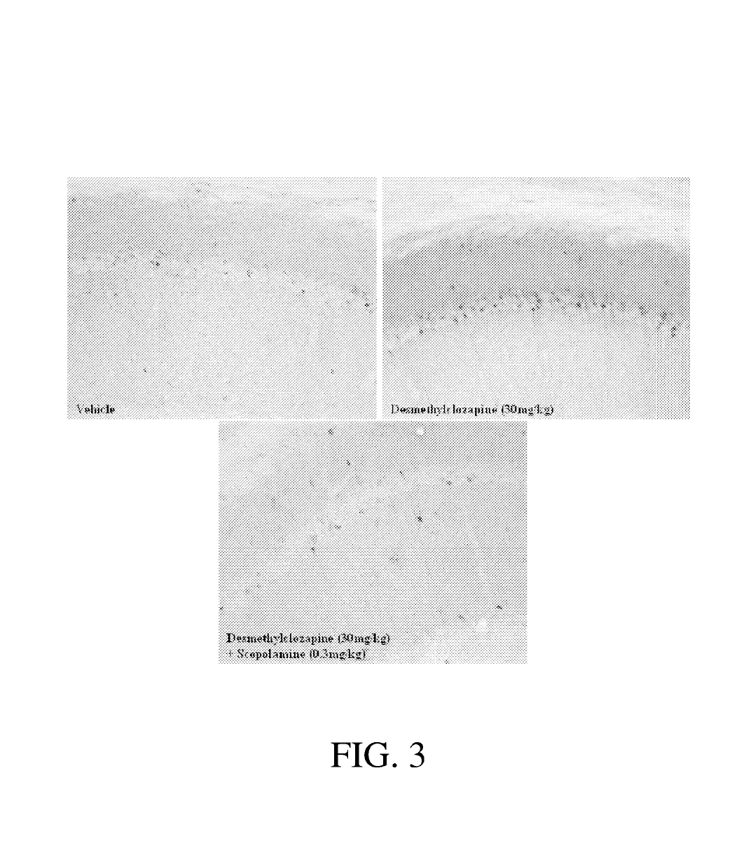

To confirm aspects of this molecular profile in vivo, and to assess the ability of NDMC to access the central nervous system, NDMC was administered parenterally to rats, and the M1 receptor mediated activation of hippocampal MAP kinase (MAPK) activity was determined, and this is disclosed in FIG. 3. NDMC treatment activates MAPK in CA1 pyramidal neurons. C57BL6 mice were treated s.c with vehicle, N-desmethylclozapine, clozapine, or NDMC and scopolamine (i.p.) at the doses described in FIG. 3, and then subjected to labeling via immunohistochemistry. With NDMC treatment, cell bodies and proximal dendrites of CA1 pyramidal neurons showed increased phospho-MAPK immunoreactivity compared to either vehicle or clozapine treatment. Furthermore, scopolamine reduced NDMC induced MAPK activation in the CA1 region indicative of a muscarinic receptor mediated mechanism. Robust activation was observed, at a dose of 30 mg/kg. This confirms that NDMC penetrates the blood brain barrier, and function as a muscarinic receptor agonist in vivo.

### Example 2

### Nonclinical Pharmacology of NDMC

A comprehensive functional pharmacological screen of nearly all known antipsychotics, and many of their metabolites, at a majority of the known biogenic amine G-protein-coupled receptors (GPCRs) identified NDMC as pharmacologically unique. NDMC is an antagonist of D₂ dopamine receptors and a potent inverse agonist of 5HT_{2A} receptors. However, unlike any other compound tested, NDMC is a potent and efficacious muscarinic receptor agonist. Specifically, NDMC is a potent partial agonist of M₁ (Kᵢ=50 nM) and M₅ receptors (Kᵢ=25 nM). NDMC also displays agonism of M₂, M₃, and M₄ receptors, however this interaction is 10-fold less potent than the interaction with other subtypes and indeed, under physiological conditions NDMC is able to competitively antagonize M₃ receptors. In comparison, clozapine is a potent competitive antagonist of M₁, M₃, and M₅ receptors, a weak agonist of M₂ receptors, and a potent partial agonist of M₄ receptors. Furthermore, olanzapine, an antipsychotic structurally related to NDMC and clozapine is an antagonist of all muscarinic subtypes. Haloperidol, risperidone, and ziprasidone do not interact with any of these receptors at concentrations up to 1 μM. Thus, the agonist activity of NDMC at muscarinic receptors, particularly M₁ and M₅ receptors, is unique among antipsychotic drugs.

In addition to its activity at D₂, 5HT_{2A}, and muscarinic receptors, NDMC has affinity for α₁, α₂, D₁, H₁, δ₂, 5HT_{1A}, 5HT_{1B}, 5HT₃, 5HT₆, and 5HT₇ receptors, and Ca²⁺ channels in ligand binding assays. Functionally it is a potent competitive antagonist of 5HT_{2C}, H₁, and α_{1A} receptors and an inverse agonist of 5HT_{6A} and 5HT_{7A} receptors.

NDMC is orally active in two models thought to be predictive of antipsychotic activity. Like clozapine, NDMC attenuates both MK-801-induced and amphetamine-induced hyperactivity in mice at doses lower or similar to those that reduce spontaneous activity. Unlike clozapine and haloperidol, NDMC does not attenuate apomorphine-induced climbing in mice. This may reflect the reduced affinity of NDMC for D₂ receptors compared to these other antipsychotics. NDMC administration results in a dose-dependent activation of mitogen-activated protein kinase (MAPK) in the CA1 region of hippocampus and this activation can be blocked by the non-selective muscarinic antagonist scopolamine. Given that M₁ receptors are the predominant subtype of muscarinic receptor responsible for MAPK activation in the CA1 region of the hippocampus, this finding supports the in vivo agonism of M₁ receptors by NDMC. Clozapine administration does not result in MAPK activation. Additional evidence of pharmacological activity of NDMC comes from the observation that NDMC administration increases cFOS expression in the prefrontal cortex and nucleus accumbens, but not in the striatum. The lack of cFOS expression in the striatum suggests that NDMC is unlikely to produce extrapyramidal side effects.

### Example 3

### Nonclinical Pharmacokinetics and Metabolism of NDMC

The pharmacokinetics of NDMC and clozapine were investigated in rats and dogs. In both species, a single dose of NDMC was administered orally (10 mg/kg) or intravenously (1 mg/kg) and blood samples were taken at regular intervals post-dose. The data showed that the oral bioavailability of NDMC is 25% and 44% in rats and dogs, respectively. In comparison, the oral bioavailability of clozapine is 1.5% and 7% in rats and dogs, respectively. Thus these data indicate that NDMC has superior oral bioavailability relative to clozapine.

In animals that received clozapine, appreciable levels of NDMC were detected. In rats, NDMC levels at Cₘₐₓ were approximately 20-fold higher than the levels of clozapine at its Cₘₐₓ. In dogs, peak NDMC levels were approximately 16% of the peak clozapine levels. These data confirm published studies that demonstrate the metabolism of clozapine to NDMC in several species including mice, rabbit, dog, pig, monkey, and human.

The brain-to-plasma ratio of NDMC was calculated in rats. The ratio was 1.0 at 240 minutes after oral administration of NDMC and 2.6 at 240 minutes after oral administration of clozapine. Together with data available in the literature, these results show that NDMC distributes into the CNS.

### Example 4

### In vitro Pharmacology of NDMC

The affinity of NDMC for 50 receptors, ion channels, and transporters was evaluated at a single high dose (10 μM). This screen identified 16 sites at which NDMC caused 90% or greater inhibition of binding and these were α₁, α₂, D₁, D_{2S}, H₁, M₁, M₂, M₃, δ₂, 5HT_{1A}, 5HT_{1B}, 5HT_{2A}, 5HT₃, 5HT₆, and 5HT₇ receptors, and Ca²⁺ channels. The inhibition of ligand binding in these assays provides information regarding the binding of NDMC to these receptors, however does not indicate the nature of the interaction.

### Example 5

### Functional Screen of NDMC Against Multiple G-Protein-Coupled Receptors (GPCRs)

The pharmacological profile of NDMC was extensively studied in a wide range of functional GPCR assays using proprietary Receptor Selection and Amplification Technology (R-SAT; 2, 3). Table 3 reports the functional pharmacological activity of NDMC and leading typical and atypical antipsychotics at a subset of human monoaminergic receptor at which these drugs demonstrate the highest potencies.

TABLE 3
Antagonist and Inverse Agonist Activity of NDMC and Reference
Antipsychotics in R-SAT Assays

| | Compound | | | | | |
|---|---|---|---|---|---|---|
| Receptor | NDMC | Clozapine | Olanzapine | Haloperidol | Risperidone | Ziprasidone |
| | Competitive Antagonist | | | | | |
| | pKi | pKi | pKi | pKi | pKi | pKi |
| D₂ | 7.2 ± 0.1 | 7.7 ± 0.1 | 8.4 ± 0.2 | 10.0 ± 0.1 | 9.3 ± 0.1 | 8.3 ± 0.3 |
| 5-HT_{2A} | 8.3 ± 0.2 | 8.3 ± 0.2 | 8.6 ± 0.1 | 7.3 ± 0.1 | 9.7 ± 0.1 | 8.6 ± 0.1 |
| 5-HT_{1A} | nr¹ | nr | nr | nr | nr | nr*² |
| 5-HT_{2C} | 7.8 ± 0.2 | 7.4 ± 0.2 | 7.4 ± 0.1 | nr | 7.2 ± 0.3 | 7.4 ± 0.2 |
| H₁ | 8.2 ± 0.2 | 9.5 ± 0.2 | 8.4 ± 0.1 | nr | 7.0 ± 0.2 | nr |
| M₁ | nr* | 7.8 ± 0.2 | 7.2 ± 0.2 | nr | nr | nr |
| M₂ | nr* | nr* | 6.9 ± 0.1 | nr | nr | nr |
| M₃ | 6.8 ± 0.7 | 8.2 ± 0.2 | 6.7 ± 0.5 | nr | nr | nr |
| M₄ | nr* | nr* | 7.4 ± 0.3 | nr | nr | nr |
| M₅ | nr* | 7.5 ± 0.3 | 7.2 ± 0.2 | nr | nr | nr |
| D₃ | nr | 6.3 ± 0.1 | 7.6 ± 0.4 | 9.7 ± 0.1 | 7.9 ± 0.4 | 7.5 ± 0.3 |
| α_{1A} | 7.3 ± 0.1 | 8.1 ± 0.1 | 7.4 ± 0.2 | 7.4 ± 0.1 | 8.5 ± 0.1 | 7.4 ± 0.2 |
| α_{2A} | nr | nr | nr | nr | 7.7 ± 0.1 | nr |
| | Inverse Agonist | | | | | |
| | pEC₅₀ | pEC₅₀ | pEC₅₀ | pEC₅₀ | pEC₅₀ | pEC₅₀ |
| 5HT_{2A} | 8.0 ± 0.3 | 8.0 ± 0.3 | 7.8 ± 0.1 | 6.8 ± 0.1 | 9.0 ± 0.3 | 8.8 ± 0.3 |
| 5HT_{6A} | 6.9 ± 0.1 | 7.0 ± 0.2 | 7.4 ± 0.2 | nr | nr | nr |
| 5HT_{7A} | 7.3 ± 0.1 | 7.4 ± 0.1 | nr | nr | 9.1 ± 0.2 | 7.3 ± 0.1 |

¹nr = no significant antagonist or inverse agonist activity up to 1 μM.
²nr* = no significant antagonist or inverse agonist activity up to 1 μM; significant agonist activity (see Table 2).

The pharmacological activity of NDMC was similar to that of existing, clinically efficacious atypical antipsychotics. Like all atypical antipsychotics, NDMC showed high potency, competitive antagonist and inverse agonist activity at 5-HT_{2A} receptors. It displayed lower potency as a dopamine D₂ receptor antagonist, than clozapine and therefore has a higher 5-HT_{2A}/D₂ receptor potency ratio. NDMC also displayed lower potency as an H₁ and α_{1A} receptor antagonist than clozapine, suggesting that it may have less of a propensity to induce adverse clinical effects, including sedation and orthostatic hypotension, mediated by these receptor subtypes. Consistent with these data, published reports confirm the potent competitive antagonist activity of NDMC at D₂ and 5-HT_{2C} receptors in vitro (Kouppamäki M, Syvalahti E and Hietala J (1993). Clozapine and N-desmethylclozapine are potent 5-HT_{1C} receptor antagonists. Eur J Pharm, 245: 179-182), the lack of potent activity at histamine H₃ receptors (Alves-Rodriques A, Leurs R. Willems E and Timmerman H (1996). Binding of clozapine metabolites and analogues to the histamine H₃ receptor in rat brain cortex. Arch Pharm Pharm Med Chem, 329: 413-416; Schlicker E and Marr 1 (1996). The moderate affinity of clozapine at H₃ receptors is not shared by its two major metabolites and by structurally related and unrelated atypical neuroleptics. Naunyn-Sch Arch Pharmacol, 353: 290-294), and only low potency interactions with GABA_{A} receptors (Wong G. Kuoppamaki M, Hietala J. Lüddens H. Syvälahti E and Korpi ER (1996). Effects of clozapine metabolites and chronic clozapine treatment on rat brain GABA_{A} receptors. Eur J Pharm, 314: 319-323).

Of the antipsychotics screened, only NDMC and clozapine possessed muscarinic receptor agonist properties (Table 2; Sur C, Mallorga P J, Wittmann M, Jacobsen M A, Pascarella D, Williams J B, Brandish P E, Pettibone D J, Scolnick E M and Conn P J (2003). N-desmethylclozapine, an allosteric agonist at muscarinic 1 receptor, potentiates N-methyl-D-aspartate receptor activity. PNAS, 100: 13674-13679). NDMC was a potent, partial agonist of human M₁ and M₅ receptors and a less potent, full agonist of human M₂ and M₄ receptors (Table 2); it lacked antagonist activity at these receptors under similar conditions (Table 1). The physiological significance of M₂ and M₅ agonism in schizophrenia is unknown. However, agonism of M₁ and M₄ receptors is associated with antipsychotic activity (Bymaster F P, Felder C, Ahmned S and McKinzie D (2002). Muscarinic Receptors as a Target for Drugs Treating Schizophrenia. Curr Drug Targ CNS Neurol Dis, 1: 163 -181; Felder C C, Bymaster F P, Ward J and DeLapp N (2000). Therapeutic Opportunities for Muscarinic Receptors in the Central Nervous System. J Med Chem, 43: 4333-4353). Furthermore, agonism of M₁ receptors may confer cognition-enhancing activity on NDMC (Bymaster F P, Felder C, Ahmned S and McKinzie D (2002). Muscarinic Receptors as a Target for Drugs Treating Schizophrenia. Curr Drug Targ CNS Neurol Dis, 1: 163-181). NDMC displays minimal, low potency agonist activity at M₃ receptors and behaves as an antagonist at this site (Tables 3 and 4). Muscarinic M₃ receptors are the predominant receptor subtype that mediate cholinergic effects of parasympathetic activation in humans, such that significant agonist activity would likely result in treatment-limiting parasympathetic side effects including sweating, ocular, and gastrointestinal dysfunction. The antagonist activity of NDMC at M₃ suggests that severe parasympathetomimetic effects will not be observed in clinical testing. The pharmacological activity of NDMC at the muscarinic receptors has been observed by others (Sur et al. PNAS 2003).

TABLE 4
Muscarinic Receptor Agonist Activity of Dibenzodiazepine Antipsychotics

| | M1 | | M2 | | M3 | | M4 | | M5 | |
|---|---|---|---|---|---|---|---|---|---|---|
| Compound | Efficacy¹ | pEC₅₀ | Efficacy | pEC₅₀ | Efficacy | pEC₅₀ | Efficacy | pEC₅₀ | Efficacy | pEC₅₀ |
| NDMC | 72 ± 5² | 7.3 ± 0.1 | 106 ± 19 | 6.5 ± 0.2 | 27 ± 4 | 6.5 ± 0.2 | 87 ± 8 | 6.9 ± 0.2 | 48 ± 6 | 7.6 ± 0.3 |
| Clozapine | 24 ± 3 | 7.3 ± 0.4 | 65 ± 8 | 6.5 ± 0.1 | nr | | 57 ± 5 | 7.4 ± 0.1 | nr | |
| Olanzapine | nr | | nr | | nr | | nr | | nr | |
| Carbachol | 101 ± 2 | 6.1 ± 0.1 | 101 ± 5 | 6.3 v 0.1 | 102 ± 3 | 6.5 ± 0.1 | 96 ± 3 | 6.5 ± 0.1 | 105 ± 3 | 6.8 ± 0.1 |

¹Efficacy is % carbachol activation of the receptor
²Data are mean ± S.E.M.
³nr = no significant agonist activity up to 10 μM

The pharmacological profile of NDMC at the muscarinic receptors is distinct from that of clozapine. Clozapine displayed potent agonist activity at M₁ receptors, however the efficacy of this interaction was very low (Table 4) and under similar conditions clozapine was a potent antagonist of M₁ receptor activation (Table 3). Also in contrast to NDMC, clozapine demonstrated potent M₃ and M₅ antagonism. At the M₂ and M₄ receptors clozapine demonstrated partial agonism. These data predict that, whereas it is likely that NDMC will behave as an M₁ agonist in vivo, clozapine is likely to act as an M₁ antagonist.

### Example 6

### Effect of NDMC on Spontaneous Locomotion and Reversal of MK-801-Induced Hyperactivity in Non-Swiss Albino Mice

NDMC was administered subcutaneously (s.c.) or orally (p.o.) to male, adult Non-Swiss Albino (NSA) mice at 1, 10, or 30 mg/kg. Upon both s.c. and p.o. administration, NDMC significantly reduced spontaneous activity at 10 and 30 mg/kg. At 10 mg/kg s.c. the maximal reduction was achieved at 30 minutes post-administration and was maintained for the duration of the experiment, 120 minutes. This effect of NDMC was similar to that seen with clozapine, which reduced spontaneous locomotion at 3 and 10 mg/kg s.c. and p.o.

Clinically effective antipsychotic drugs can block the behavioral effects of non-competitive N-methyl-D-aspartate agonists, such as MK-801. NDMC was evaluated for its ability to attenuate MK-801-induced hyperactivity in male, adult, NSA mice and its activity in this assay was compared to that of clozapine. NDMC attenuated MK-801-induced hyperactivity with a minimal effective dose of 1 mg/kg s.c. and 10 mg/kg p.o., consistent with antipsychotic-like efficacy. These doses were lower than or similar to those that reduced spontaneous locomotion, suggesting that the antipsychotic-like effects can be differentiated from general locomotor behavioral disruption. Similarly, clozapine reduced MK-801-induced hyperactivity with a minimal effective dose of 1 mg/kg s.c. and 3 mg/kg p.o.

### Example 7

### Effect of NDMC on the Reversal of Amphetamine-Induced Locomotor Behaviors in Non-Swiss Albino Mice

Similar to attenuation of hyperactivity induced by N-methyl-D-aspartate agonists, clinically effective antipsychotics also attenuate dopamine-mediated hyperactivity in rodents. Amphetamine-induced hyperactivity in mice is, therefore, a commonly used assay for in vivo antipsychotic-like activity. NDMC attenuated amphetamine-induced hyperactivity in male, adult NSA mice at 10 mg/kg after s.c. or p.o. administration. Clozapine also reduced amphetamine-induced hyperactivity with a minimal effective dose of 3 mg/kg p.o. These results are predictive of antipsychotic-like efficacy in humans.

### Example 8

### Effect of NDMC on Reversal of Apomorphine-Induced Climbing in Non-Swiss Albino Mice

Another way to assess the blockade of dopamine-mediated behavior in rodents is the attenuation of apomorphine-induced climbing in mice. Direct D₂ receptor antagonists most effectively block climbing induced by the dopamine receptor agonist apomorphine. Haloperidol, a typical neuroleptic antipsychotic drug with high affinity for dopamine D₂ receptors, completely attenuated the apomorphine-induced climbing in male, adult, NSA mice at 0.1 mg/kg s.c. Clozapine also reduced apomorphine-induced climbing in a dose-dependent manner with the minimal effective dose at 10 mg/kg s.c. In contrast NDMC did not attenuate apomorphine-induced climbing at doses up to 100 mg/kg s.c. This may reflect the reduced affinity of NDMC for D₂ receptors as compared to clozapine and haloperidol.

### Example 9

### Effect of NDMC on MAPK Activation in Brain in C57BL/6 Mice

In an effort to confirm the muscarinic agonist properties of NDMC in vivo, the activation of mitogen-activated protein kinase (MAPK) in CA1 region of the hippocampus was examined. NDMC was administered s.c. at doses of 3, 10, 30, and 100 mg/kg to C57BL/6 mice. The animals were killed two hours later; whole brains were removed and subjected to immunodetection of MAPK activity in hippocampus. NDMC administration resulted in the stimulation of MAPK activity at all doses in a dose-dependent manner. In contrast, clozapine at 30 mg/kg did not result in MAPK activation in CA1 region of brain. The stimulation of MAPK activity induced by NDMC was blocked by the non-selective muscarinic receptor antagonist scopolamine (0.3 mg/kg, i.p.), confirming that NDMC acts as a muscarinic receptor agonist in vivo. It has been demonstrated in vitro that M₁ receptors are the predominant subtype of muscarinic receptor that is responsible for activation of MAPK in the forebrain (Hamilton S E and Nathanson N M (2001). The M₁ Receptor is required for Muscarinic Activation of Mitogen-activated Protein (MAP) Kinase in Murine Cerebral Cortical Neurons. J Biol Chem, 276: 15850-15853; Berkeley J L, Gomeza J. Wess J. Hamilton S E, Nathanson N M and Levey A I (2001). M₁ Muscarinic Acetylcholine Receptors Activate Extracellular Signal-Regulated Kinase in CAI Pyramidal Neurons in Mouse Hippocampal Slices. Mol Cell Neurosci, 18: 512-524; Berkeley J L and Levey A I (2003). Cell-Specific Extracellular Signal-regulated Kinase Activation by Multiple G Protein-coupled receptor Families in Hippocampus. Mol Pharm, 63: 128-135). Hence these data support the in vivo agonism of muscarinic M₁ receptors by NDMC.

### Example 10

### Effects of Desmethylclozapine on Fos Protein Expression in the Forebrain: In vivo Biological Activity of the Clozapine Metabolite

The first in vivo demonstration of pharmacological activity of NDMC (desmethylclozapine) was a dose-dependent induction of the expression of the immediate early gene cFOS in rat brain (Young C D, Meltzer H Y and Deutch A Y (1997). Effects of desmethylclozapine on Fos protein expression in the forebrain: In vivo biological activity of the clozapine metabolite. Neuropsychopharm, 19: 99-103). NDMC was administered to adult male Sprague-Dawley rats s.c. at doses of 7.5 and 30.0 mg/kg; the animals were sacrificed two hours later and homogenized tissue from various brain regions was subjected to immunodetection of cFOS by western blotting. NDMC resulted in the induction of cFOS expression in the pre-frontal cortex and nucleus accumbens, but not in striatum, and these effects were similar in magnitude and regional selectivity to those observed for clozapine. The lack of cFOS expression in the striatum of NDMC-treated animals may indicate a low propensity for NDMC to cause EPS.

### Example 11

### Pharmacokinetic Evaluation of Clozapine and N-Desmethylclozapine Following Administration of a Single Intravenous Dose or Oral Dose to Conscious Sprague Dawley Rats

The pharmacokinetics of clozapine and N-desmethylclozapine (NDMC) was evaluated in rats after intravenous (i.v.) and oral (p.o.) dosing. Cₘₐₓ, Tₘₐₓ and bioavailability after p.o. dosing and the volume of distribution (Vss), terminal plasma half-life (T_{1/2}) and clearance (CLs) after i.v. dosing were determined. The brain-to-plasma ratio of NDMC after both intravenous and oral administration was also determined. A total of 18 male Sprague-Dawley rats were dosed with clozapine p.o. (N=6, 10 mg/kg), NDMC p.o. (N=6, 10 mg/kg), clozapine i.v. (N=6, 1 mg/kg), or NDMC i.v. (N=6, 1 mg/kg), and serum samples for bioanalytical analysis were obtained at regular intervals at between 0 and 240 minutes post dose. Animals were euthanised and brain and plasma samples obtained at 60 or 240 minutes post-dose, depending on study group. The levels of NDMC and clozapine were measured in each sample. Pharmacokinetic data for NDMC is presented in tables 5-8.

TABLE 5
Plasma Concentration (ng/mL¹) of NDMC in Rat after NDMC
Administration²

| Compound | Time (min) | | | | | |
|---|---|---|---|---|---|---|
| Measured (route) | 10 | 30 | 60 | 120 | 180 | 240 |
| NDMC (p.o.) | 305 ± 101 | 582 ± 265 | 481 ± 181 | 227 ± 75 | 170 ± 26 | 122 ± 54 |
| NDMC (p.o.) | 277 ± 57 | 576 ± 161 | 614 ± 60 | NS³ | NS | NS |
| NDMC (i.v.) | 540 ± 46 | 276 ± 30 | 126 ± 38 | 33.7 ± 11.4 | 11.7 ± 3.8 | 5.3 ± 0.3 |

¹Mean ± SD;
²Dosages for oral administration were 10 mg/kg and 1 mg/kg for intravenous administration;
³NS = no sample taken because study terminated at 60 minutes

TABLE 6
Plasma Concentration (ng/mL¹) of NDMC and Clozapine in Rat after
Clozapine Administration²

| Compound | Time (min) | | | | | |
|---|---|---|---|---|---|---|
| Measured (route) | 10 | 30 | 60 | 120 | 180 | 240 |
| Clozapine (p.o.) | 3.8 ± 1.5 | 10.2 ± 5.2 | 10.8 ± 6.0 | 5.2 ± 2.0 | 2.8 ± 0.8 | 2.2 ± 0.3 |
| Clozapine (p.o.) | 4.9 ± 1.7 | 35.8 ± 30.8 | 38.0 ± 39.0 | NS³ | NS | NS |
| Clozapine (i.v.) | 112⁴ | 75.1 ± 6.3 | 44.5 ± 4.0 | 24.8 ± 1.8 | 13.6 ± 2.6 | 9.5 ± 1.5 |
| NDMC (p.o.) | 77.1 ± 88.7 | 194 ± 161 | 147 ± 86.6 | 42.5 ± 15.1 | 13.4 ± 2.54 | 7.1 ± 0.5 |
| NDMC (p.o.) | 241 ± 21.3 | 576 ± 135 | 510 ± 247 | NS | NS | NS |
| NDMC (i.v.) | 3.5⁴ | 2.8 ± 1.2 | 4.0 ± 1.5 | 2.3 ± 1.0 | 0.7 ± 0.1 | 0.8 ± 0.6 |

¹Mean ± SD;
²Dosages for oral administration were 10 mg/kg and 1 mg/kg for intravenous administration;
³NS = no sample taken because study terminated at 60 minutes;
⁴N = 2

TABLE 7
Pharmacokinetic Parameters¹ of NDMC in Rat after NDMC Administration

| | Average | | | | | | |
|---|---|---|---|---|---|---|---|
| Compound | AUC | Cₘₐₓ | Tₘₐₓ | T_{1/2} | BA² | Vss | CLs |
| Measured (route) | (min · ng⁻¹ · mL⁻¹) | (ng/mL) | (min) | (min) | (%) | (L/kg) | (mL · min⁻¹ · kg⁻¹) |
| NDMC (i.v.) | 27331 | 756 | 0 | 39.3 | — | 1.47 | 36.2 |
| NDMC (p.o.) | 68227 | 582 | 60 | ND³ | 25.0 ± 7.5 | ND | ND |

¹Mean ± SD;
²BA = oral bioavailability;
³ND = not determined

TABLE 8
Pharmacokinetic Parameters¹ of NDMC and Clozapine in Rat after
Clozapine Administration

| | Average | | | | | | |
|---|---|---|---|---|---|---|---|
| Compound | AUC | Cₘₐₓ | Tₘₐₓ | T_{1/2} | BA² | Vss | CLs |
| Measured (route) | (min · ng/mL) | (ng/mL) | (min) | (min) | (%) | (L/kg) | (mL · min⁻¹ · kg⁻¹) |
| NDMC (i.v.) | 489.7 | 3.99 | 60 | — | — | — | — |
| NDMC (p.o) | 16199 | 194 | 30 | — | — | — | — |
| Clozapine (i.v.) | 8836 | 137 | 0 | 79.4 | — | 9.88 | 101 |
| Clozapine (p.o.) | 1347 | 10.8 | 60 | ND³ | 1.5 ± 0.6 | ND | ND |

¹Mean ± SD;
²BA = oral bioavailability;
³ND = not determined

These data demonstrate that NDMC was rapidly absorbed from the gastrointestinal tract following oral administration; a Cₘₐₓ of 582 ng/nL was achieved by 30 minutes. NDMC had low clearance from the circulation, a low volume of distribution, and was approximately 25% orally bioavailable. Clozapine reached much lower peak drug levels (10.8 ng/mL; 1/50^{th} that of NDMC), had higher clearance, and poorer bioavailability (1.5%) following oral administration. These data suggest that NDMC may have acceptable pharmacokinetic properties after oral administration in humans and may indeed have improved pharmacokinetic properties as compared to clozapine.

High plasma levels of NDMC were observed following oral administration of clozapine and peak plasma levels of NDMC were nearly 20-fold greater than those observed for clozapine (194 ng/nL versus 10.8 ng/nL). Similar observations have been made by others (Weigmann H. Härter S. Fischer V, Dahmen N and Hiemke C (1999). Distribution of clozapine and desmethylclozapine between blood and brain in rats. Eur Neuropsychopharm, 9: 253-256; Baldessarini R J, Centorrino F, Flood J G, Volpicelli S A, Huston-Lyons D and Cohen B M (1993). Tissue concentrations of clozapine and its metabolite in the rat. Neuropsychopharm, 9: 117-124). Weigmann et al. (Eur Neuropsychopharm 1999) showed that following oral administration of 5 doses (20 mg/kg) of clozapine at 1.5-hour intervals to male Sprague-Dawley rats, plasma concentrations of NDMC exceeded those of clozapine by up to 2.2-fold. In another study, high levels of circulating NDMC were observed following intraperitoneal (i.p.) administration of varying (1-60 mg/kg) doses of clozapine to Sprague-Dawley rats (Baldessarini et al; Neuropsychopharm 1993). Thus, NDMC is a major chemical moiety formed after oral administration of clozapine in the rat. It is also been shown in vitro that NDMC is the primary clozapine metabolite formed by rat liver microsomes (Bun H, Disdier B, Aubert C and Catalin J (1999). Interspecies variability and drug interactions of clozapine metabolism by microsomes. Fund Clin Pharm, 13: 577-581).

The pharmacokinetic study described above included an initial assessment of the distribution of NDMC into brain. The ratio of brain-to-plasma levels of NDMC was 0.36±0.16 at 60 minutes and 1.0±0.4 at 240 minutes following oral administration of 10 mg/kg NDMC to Sprague-Dawley rats. Additionally, after oral administration of clozapine the brain-to-plasma ratio of NDMC was 0.26±0.07 at 60 minutes and 2.6±0.8 at 240 minutes. This latter result confirms previously published findings showing that oral administration of clozapine to male Sprague-Dawley rats resulted in NDMC levels in brain that were up to 3.9-fold higher than those observed in serum (Baldessarini et al.; Neuropsychopharm 1993) and intraperitoneal administration of 20, 30, and 60 mg/kg of clozapine to Sprague-Dawley rats resulted in the detection of NDMC in brain (Bun et al.; Fund Clin Pharm 1999). Together these in vivo data clearly document that NDMC distributes into the CNS after oral administration.

### Example 12

### Bioavailability Assessment of Clozapine and N-Desmethylclozapine in Male Beagle Dogs

The pharmacokinetics of clozapine and N-desmethylclozapine (NDMC) were evaluated in dogs after intravenous (i.v.) and oral (p.o.) dosing. Cₘₐₓ, Tₘₐₓ and bioavailability after p.o. dosing and the volume of distribution (Vss), terminal plasma half-life (T_{1/2}) and clearance (CLs) after i.v. dosing were determined. A total of 6 beagle dogs were dosed with clozapine p.o. (N=3, 10 mg/kg), NDMC p.o. (N=3, 10 mg/kg), clozapine i.v. (N=3, 1 mg/kg), or NDMC i.v. (N=3, 1 mg/kg). Serum samples for bioanalytical analysis were obtained pre-dose and 10 min, 30 min, 1, 2, 3, 4, and 6 h post dose after p.o. administration and pre-dose, 2, 5, 10, 30 min, 1, 2 3, and 4 h after i.v. administration. The levels of NDMC and clozapine were measured in each sample. Pharmacokinetic data for NDMC are presented in tables 9-12.

TABLE 9
Plasma Concentration (ng/mL¹) of NDMC in Dog after NDMC
Administration²

| Compound | | | | | | | | |
|---|---|---|---|---|---|---|---|---|
| Measured (route) | Time (min) | | | | | | | |
| | — | 10 | 30 | 60 | 120 | 180 | 240 | 360 |
| NDMC (p.o.) | — | 1.0 | 14 ± 12² | 67 ± 37 | 155 ± 95 | 249 ± 44 | 274 ± 44 | 261 |
| | 2 | 5 | 10 | 30 | 60 | 120 | 180 | 240 |
| NDMC (i.v.) | 182.5 ± 90 | 73 ± 22 | 50 ± 10 | 35 ± 2 | 32 ± 6 | 28 ± 4 | 27 ± 7 | 27 ± 4 |

¹Mean SD;
²Dosages for oral administration were 10 mg/kg and 1 mg/kg for intravenous administration.

TABLE 10
Plasma Concentration (ng/mL¹) of NDMC and Clozapine in Dog after Oral
of Intravenous Clozapine Administration²

| Compound | | | | | | | | |
|---|---|---|---|---|---|---|---|---|
| Measured (route) | Time (min) | | | | | | | |
| | — | 10 | 30 | 60 | 120 | 180 | 240 | 360 |
| NDMC (p.o.) | — | 0 | 2.45 | 25.4 | 5.8 | 10.29 | 19.23 | 46.7 |
| Clozapine (p.o.) | | 0.46 | 9.53 | 61.8 ± 103 | 35 ± 20 | 57 ± 16 | 100 ± 33 | 213 ± 91 |
| | 2 | 5 | 10 | 30 | 60 | 120 | 180 | 240 |
| NDMC (i.v.) | 0.54 ± 0.12 | 0.47 ± 0.06 | 0.64 ± 0.26 | 1.72 ± 0.75 | 3.55 ± 1.03 | 4.31 ± 1.34 | 4.89 ± 1.41 | 4.44 ± 1.31 |
| Clozapine (i.v.) | 166 ± 28 | 136 ± 40 | 98 ± 24 | 75 ± 10 | 76 ± 7 | 61 ± 8 | 58 ± 11 | 41 ± 6 |

¹Mean SD;
²Dosages for oral administration were 10 mg/kg and 1 mg/kg for intravenous administration.

TABLE 11
Pharmacokinetic Parameters¹ of NDMC in Dog after Oral or Intravenous
NDMC and Clozapine Administration

| | Average | | | | | | |
|---|---|---|---|---|---|---|---|
| Compound | AUC | Cₘₐₓ | Tₘₐₓ | T_{1/2} | BA² | Vss | CLs |
| Measured (route) | (min · ng⁻¹ · mL⁻¹) | (ng/mL) | (min) | (min) | (%) | (L/kg) | (mL · min⁻¹ · kg⁻¹) |
| NDMC (i.v.) | 134.8 ± 21.3 | 353.2 ± 242 | — | 13.2 ± 7.0 | — | 28202.1 ± 4919.8 | 1850 ± 1060.4 |
| NDMC (p.o.) | 597.6 ± 111.8 | 286.3 ± 25 | 3.3 ± 1.2 | ND | 44.3 | ND | ND |
| Clozapine (i.v.) | 15.0 ± 3.9 | 5.3 ± 1.2 | 2.7 ± 0.58 | | | | |
| Clozapine (p.o.) | 32.1 ± 24.0 | 19.2 ± 7.2 | 4.0 ± 0.0 | | | | |

¹Mean ± SD;
²BA = oral bioavailability

TABLE 12
Pharmacokinetic Parameters¹ of Clozapine in Dog after Clozapine
Administration

| | Average | | | | | | |
|---|---|---|---|---|---|---|---|
| Compound | AUC | Cₘₐₓ | Tₘₐₓ | T_{1/2} | BA² | Vss | CLs |
| Measured (route) | (min · ng⁻¹ · mL⁻¹) | (ng/mL) | (min) | (min) | (%) | (L/kg) | (mL · min⁻¹· kg⁻¹) |
| Clozapine (i.v.) | 266 ± 33 | 189 ± 18 | — | 3.3 ± 0.63 | — | 10335 ± 1636 | 2190 ± 295.9 |
| Clozapine (p.o.) | 186 ± 109.5 | 124.9 ± 58.3 | 3.0 ± 1.7 | ND | 7.0 | ND | ND |

¹Mean ± SD;
²BA = oral bioavailability

NDMC was absorbed from the gastrointestinal tract following oral administration with a Cₘₐₓ of 286.3 ng/mL achieved by 3.3 h. NDMC had low clearance from the circulation, a low volume of distribution, and was approximately 44% orally bioavailable. Clozapine had poorer oral bioavailability (7%). These data suggest that NDMC may have acceptable pharmacokinetic properties after oral administration in humans and may indeed have improved pharmacokinetic properties as compared to clozapine.

NDMC was readily detectable in plasma following both intravenous and oral administration of clozapine. The mean NDMC/clozapine AUC ratio was 0.056 after i.v. administration of clozapine and 0.161 (i.e., 16%) after oral administration. These data confirm recent studies that demonstrated the metabolism of clozapine to N-desmethylclozapine in dog both in vitro (Bun et al. Fund Clin Pharm 1999) and in vivo (Mosier K E, Song J. McKay G. Hubbard J W and Fang J (2003). Determination of clozapine, and its metabolites, N-desmethylclozapine and clozapine N-oxide in dog plasma using high-performance liquid chromatography. J Chromat B, 783: 377-382). Mosier and colleagues showed that following oral administration of clozapine to a dog the Cₘₐₓ of desmethylclozapine was approximately 20% that of clozapine (i.e., the NDMC/clozapine ratio was approximately 0.2). An early study did not detect N-desmethylclozapine in dog (Gauch R and Michaelis W (1970)). The metabolism of 8-chloro-11-(4-mehtyl-1-piperazinyl)-5H-dibenzo[b,e][1,4]diazepine (Clozapine) in mice, dogs, and human subjects. II Farmaco, 26: 667-681) after oral administration; however this may have been due to insensitive analytical techniques.

### Example 13

### The Role of M1 Muscarinic Receptor Agonism of N-Desmethylclozapine in the Unique Clinical Effects of Clozapine

Methods

Molecular profiling of clinically relevant drugs was performed at all known monoaminergic receptor subtypes except the Dopamine D₄, Serotonin 5_{A}, and Histamine H₄ receptors using Receptor Selection and Amplification Technology (R-SAT) assays. Briefly, NIH/3T3 cells plated at 70-80% confluency were transfected with various receptor cDNA (10-100 ng receptor and 20 ng β-Gal reporter/well of a 96 well plate) using the Polyfect Reagent (Qiagen Inc.) as described in the manufacture's protocol. One day after transfection, ligands were added in Dulbecco's modified Eagle's medium supplemented with penicillin (100 U/ml), streptomycin (100 μg/ml) and 2% Cyto-SF3. After four to six days, the media was aspirated off, the cells were lysed, O-Nitrophenyl-beta-D-Galactopyranoside (ONPG) was added and the resulting absorbance was measured spectrophotometrically. Concentration response curves were performed as eight-point concentration response experiments run in duplicate, where the maximal antipsychotic concentrations varied from 10-25 micromolar, and data were analyzed using Excel fit and Graph Pad Prism. Reported EC₅₀ values represent the concentration of a ligand that produces a half-maximal response from a receptor in the absence of other ligands, and IC₅₀ values represent the concentration of a ligand that inhibits half of the agonist-induced activity. Competitive antagonist IC₅₀ data were adjusted for agonist occupancy using the equation Ki=IC₅₀/{1+[agonist]/EC₅₀ agonist}. Data are reported as negative log values (pEC₅₀ and pKᵢ). Sources of the drugs utilized in this study are described in Weiner et al. (2001) and Wellendorph et al. (2002), with the exception of N-desmethylclozapine, which was acquired from Sigma, Inc., and N-desmethylolanzapine, which was synthesized by ACADIA Pharmaceuticals. A list of the compounds screened can be found as supplemental information.

PI hydrolysis assays were performed on Chinese Hamster Ovary cells stably transfected with the human M1 muscarinic receptor cDNA as described in Spalding et al (2002), and the data are derived from six or eight-point concentration response experiments performed in duplicate.

MAP Kinase assays utilized C57BL6 mice treated subcutaneously with either vehicle, clozapine, or N-desmethylclozapine with or without scopolamine, sacrificed two hours later, and phospho-MAPK immunoreactivity was assayed as described in Berkeley et al (2001). Briefly, after treatments which were administered s.c. at 60 min., mice were perfused with 100 ml of 4% paraformaldehyde followed with 100 ml of 10% sucrose. Brains were removed and cryoprotected in 30% sucrose overnight at 4° C. The next day, 50 μm slices were cut on a sliding microtome. Slices were rinsed, treated with 3% H₂O₂ for 10 minutes at room temperature and rinsed again. Slices were blocked in PBS containing 10 μg/ml avidin (Vector Laboratories Burlingame, Calif.), 0.1% triton-X and 4% normal goat serum (NGS) for 1 hour. Slices were rinsed and incubated in PBS containing 50 μg/ml biotin (Vector Laboratories Burlingame, Calif.), 2% NGS, and phospho-ERK1/2 antibody (Cell signal Technologies, Beverly, Mass.) at a concentration of 1:250 and allowed to incubate overnight at 4° C. The next day, slices were rinsed and placed in PBS containing 2% NGS and biotinylated goat anti-rabbit (Vector Laboratories Burlingame, Calif.) at a concentration of 1:100 for 1 hour at 4° C. Slices were rinsed and placed in horseradish peroxidase-conjugated avidin-biotin complex (Vector Laboratories Burlingame, Calif.) for 1 hour at 4° C. Slices were rinsed and incubated in TSA Fluorescein tyramide for 10 min at room temperature. Slices were treated with 10 mM CuSO₄ for 30 minutes, mounted onto glass slides with Vectashield mounting media (Vector Laboratories Burlingame, Calif.). Slides were visualized via a fluorescence microscope and digital images were analyzed with Scion image analysis software (Scion Corp. Frederick, Md.).

Stepwise multiple-regression analysis, including the dependent measure, dose, age, and gender was utilized to assess the contribution of NDMC to treatment response in schizophrenic subjects (Hasegawa et al 1993 and Lee et al 1999). The analysis was adjusted for baseline level of symptom severity, age, and dose, since dose was not fixed. The plasma samples chosen for the analyses were obtained at 6 weeks and 6 months after initiation of therapy, were related to the clinical measures obtained at those times, and were drawn 12 hours after the last clozapine dose. Only subjects who had received at least 100 mg of clozapine per day were included in the analysis, and some data were unavailable for these subjects at some time points. Regarding co-treatment with anticholinergic agents, only two subjects in this sample were treated with benztropine. The results did not differ when data from these two subjects were omitted (data not shown). Lastly, ten of the patients in this study were treated with benzodiazepines at the time the levels of clozapine and NDMC were measured. Benzodiazepines have not been reported to affect the metabolism of clozapine.

Drugs screened, grouped according to clinical class, included:

Antipsychotics: Amoxapine, Amisulpiride, Amperozide, Bromperidol, Butaclamol, Chlorproethazine, Chlorpromazine, Chlorprothixene, Cis-flupentixol, Clothiapine, Clozapine, Droperidol, Fananserin, Fluphenazine, Fluspiriline, Haloperidol, Loxapine, Mazapertine, M100907, Melperone, Mesoridazine, Molindone, N-Desmethyl Clozapine, N-desmethylolanzapine, Ocaperidone, Octoclothepin, Olanzapine, Perazine, Perlapine, Pimozide, Pimpamperone, Promazine, Prothypendyl, Quetiapine, Remoxipride, Risperidone, Sertindole, Spiperone, Sulpride, Sultopride, Telfludazine, Thioridazine, Thiothixene, Tiapride, Moperone, Tiospirone, Trans-flupentixol, Trifluoperazine, Trifluoperidol, Triflupromazine, and Ziprasidone.

Antidepressants/Anxiolytics: Acetyltryptophan, Acetyltryptophanamide, Alaprocate, Alprazolam, Amitriptyline, Barbital, Bromazepam, Buproprion, Buspirone, Chloral Hydrate, Clobazam, Clonazepam, Clomipramine, Clorgyline, Chlordiazepoxide, Chlormezanone, Continine, Compazine, Desipramine, Deprenyl, Desmethyldiazepam, Diazoxide, Doxepin, Flumazenil, Flunitrazepam, Fluoxetine, Flurazepam, Fluvoxamine, Imipramine, Indatraline, Iproniazid, Maprotiline, Meprobamate, Milnacipram, Minaprine, Mirtazepine, Modafinil, Nitrazepam, Nomifensine, Nortriptyline, Oxazepam, Pargyline, Phenelzine, Prazepam, Protripytline, Rolipram, Tracazolate, Tranylcypromine, Trazadone, Triazolam, Trihexaphendyl, Trimipramine, Viloxazine, Zimelidine, Zolpidem, and Zopiclone.

CNS Miscellaneous: 3PPP, 5-Aminopentanoic Acid, 5-Hydroxy MDA, 5-Methoxy DMT, 5-Methoxytryptamine, Acetaminophen, Acetylsalicylic Acid, Alprenelol, Amantadine, Amiodarone, AMPA, Apocodeine, Apomorphine, Atropine, Baclofen, Balperidone, Benztropine, Bicuculline, Bradykinin, Bretylium, BRL 37344, Bromocriptine, Cannabidiol, Carbemazepine, Carbidopa, Cyproheptadine, Cirazoline, D-Amphetamine, (D-Ser2)-Leu Enkephalin-Thr, (Leu 5) Enkephalin, D-Phenylalanine, Dibucaine, Diclofenac, Dihydroergotamine, DOI, Domperidone, Ebalzotan, Edrophonium, Ephedrine, Etadolac, Ethosuxamide, Felbamate, Fenbufen, GABA, Gabaxadol, Galanthamine, Gamma-Vinyl GABA, Gabapentin, (−) GMC III, (+) GMC III, Heroin, Himbacine, I-4-AA, ICI 204448, Indoprofen, Isoguvacine, Ketamine, Ketaprofen, Labetalol, Lamotrigine, Levallorphan, Lidocaine, Lisuride, L-745-870, Melatonin, Metoclopromide, Memantine, Mescaline, Naftopidil, Nalbuphine, N-Allyl SKF 38393, Naloxone, Naltrexone, Naltrindole, Neostigmine, Nicotine, Nipecotic Acid, N-Methyl ICI 118-551, N-Methyldopamine, N,N-Dimethyl MDA, Norapomorphine, Norcodeine, Norfenfulramine, Normetazocine, Oxethazine, Pemoline, Pergolide, PCP, Phaclofen, Phenacetin, Phenteramine, Phenoxybenzamine, Phenytoin, Physostigmine, P-Iodoclonidine, Pirenzepine, Prilocaine, Primodone, Procaine, Prochlorperazine, Propranolol, Pseudoephedrine, Quinpirole, Raclopride, Rauwolscine, Reserpine, Rimcazole, RO-05-3663, RS 100329, RX 821002, Saclofen, Salicylamide, SCH 12679, SCH 23390, Scopolamine, SKF 81297, SKF 38393, SKF 82948, SKF 82957, SKF 83566, SR 141716A, SR 144528, Succinylcholine, Tenoxicam, Terguride, Tetracaine, Tolazoline, Tropicamide, UK 14304, Valproate, Vigabatrin, WIN 55212-2, Xylazine, Yohimbine, and Zomepirac.

Monoaminergic: 7-OH-DPAT, 8-0H-DPAT, Alpha Methyl Serotonin, Arecoline, Astemizole, Bethanacol, Carbachol, CGS 12066A, Cinanserin, Chlorpheniramine, Cimetidine, Clobenpropit, CPP, Dihydroergocristine, Dimaprit, Diphenhydramine, Doxylamine, Eltoprazine, Famotidine, Histamine, Imetit, Isomaltane, Ketanserin, Loperamide, L-Tryptophan, LY 53857, mCPP, Mesulergine, Metergoline, Methergine, Methiothepin, Methysergide, Mexamine, Mianserin, MK 212, Mepyramine, Pheniramine, Phenylbiguanide, Pimethixene, Piperazine, Pirenpirone, Prazosin, Promethazine, Pyrilamine, Quiapazine, Ranitidine, Ritanserin, SB 204741, SB 206553, Serotonin, Spiroxatrine, Sumitriptan, Thioperamide, Tripellenamine, Triprolidine, and WB 4101.

Cardiovascular: Acetazolamide, Adenosine, Albuterol, Atenolol, Amiloride, Amrinone, Bepridil, Caffeine, Catopril, CGS-15943, CGS-21680, CGP-12177A, Chlorothiazide, Clonidine, Debrisoquin, Digitoxin, Digoxin, Diltiazem, Dipyridamole, Disopyramide, Dobutamine, Doxazosin, DPCPX, Epinephrine, Enalapril, Flunarizine, Furosemide, Guanabenz, Guanethidine, Hydralazine, Hydrochlorothiazide, Isoproterenol, Isosorbide, Lidocaine, Linisopril, Metaproterenol, Methoxamine, Metrifudil, Metolazone, Metoprolol, Midodrine, Minoxidil, N-Acethylpocainamide, Nicardipine, Nifedipine, Nimodipine, Nitrendipine, Norepinephrine, Nylidrin, Oxymetazoline, Paraxanthine, Pentoxifylline, Phentolamine, Pinacidil, Pindolol, Procainamide, Propranalol, Quinidine, Spironolactone, Theophylline, Theoyphylline 1-3, Timolol, Triamterene, Urapidil, Verapamil, and Warfarin.

Systemic Miscellaneous: Acyclovir, Adephenine, Allupurinol, Amodiaquine, 6-bromo-APB, Artemisinin, Azathioprine, Azithromycin, Camphor, Capsaicin, Carbetapentane, Carisoprodol, Cefotaxime, Cinchonidine, Chloramphenicol, Chloroquine, Chlorpropamide, Chlorzoxazone, Clarithromycin, Clofilium, Clotrimazole, Cyclobenzaprine, D-Cycloserine, Danazol, Dantrolene, Dextromethorphan, Dimethadione, Dropropizine, E-Capsaicin, Edoxudine, Ethinimate, Fipexide, Fluconazole, Foscarnet, Gallamine, Glibenclamide, Glipizide, Hypericin, Ibuprofen, Ifenprodil, Indomethacin, Isobutylmethylxanthine, Kainic Acid, Ketoconazole, Levorphanol, Linopiridine, Mazindol, Meclizine, Mefexamide, Mefloquine, Mephenesin, Mesbeverine, Methocarbamol, Metoclopramide, Metronidazole, MK 801, N-Aminohexyl-5-Chloronaphthalene-1-Sulfonamide, N-Methyl-D-Aspartic Acid, NCS 382, Neophesperidin, Nixoxetine, Nocapine, Octopamine, Omeprazole, Orphenadrine, Oxyphenbutazone, Papaverine, Penicillamine, Pentamidine, Phenacemide, Picrotoxin, Pitrazepine, Piracetam, Piroxicam, Primaquine, Probenecid, Pyrimethamine, Quinine, Ritodrine, Saccharin, Sulindac, Suramin, SB 218795, Thalidomide, Tilorone, Trimeprazine, Tolazamide, Tolbutamide, Tolperisone, Uridine, Vidarabine, Zaleplon, and Zidovudine.

Results and Discussion

A library of 462 clinically relevant drugs were profiled for functional activity at 33 of the 36 known human monoaminergic G-protein coupled receptors using the mammalian cell-based functional assay Receptor Selection and Amplification Technology (R-SAT). Table 13 illustrates data on representative antipsychotic agents for receptors at which the most potent activities were observed. Potency data for five representative antipsychotics and the clozapine metabolite N-desmethylclozapine (NDMC) at 13 human monoamine receptor subtypes are shown. Potency data are reported as pKi values for the competitive antagonist studies, while inverse agonist data are reported as pEC₅₀ values, both derived from three to eight separate determinations ± standard error. Asterixes (*) indicate the presence of agonist activity where the muscarinic receptor agonist potencies are reported in Table 14. Ziprasidone displays limited but detectable agonist efficacy at human 5-HT_{1A} receptors (<30% relative to 8-OH-DPAT), and a Ki >1-micromolar when assayed as a competitive antagonist. Abbreviations used: NDMC-N-desmethylclozapine, 5-HT-serotonin, H-histamine, M-muscarinic, D-dopamine, and Alpha-alpha adrenergic, and nr-no response defined as no significant antagonist or inverse agonist activity at concentrations up to 1-micromolar.

TABLE 13
Pharmacological activities of antipsychotics at human monoamine receptors.

| | Haloperidol | Risperidone | Ziprasidone | Olanzapine | Clozapine | NDMC |
|---|---|---|---|---|---|---|
| | Competitive Antagonist | | | | | |
| Receptor | pKi | pKi | pKi | pKi | pKi | pKi |
| D2 | 10.0 +/− 0.1 | 9.3 +/− 0.1 | 8.3 +/− 0.3 | 8.4 +/− 0.2 | 7.7 +/− 0.1 | 7.2 +/− 0.1 |
| 5-HT2A | 7.3 +/− 0.1 | 9.7 +/− 0.1 | 8.6 +/− 0.1 | 8.6 +/− 0.1 | 8.3 +/− 0.2 | 8.3 +/− 0.2 |
| 5-HT1A | nr | nr | nr* | nr | nr | nr |
| 5-HT2C | nr | 7.2 +/− 0.3 | 7.4 +/− 0.2 | 7.4 +/− 0.1 | 7.4 +/− 0.2 | 7.8 +/− 0.2 |
| H1 | nr | 7.0 +/− 0.2 | nr | 8.4 +/− 0.1 | 9.5 +/− 0.2 | 8.2 +/− 0.2 |
| M1 | nr | nr | nr | 7.2 +/− 0.2 | 7.8 +/− 0.2 | nr* |
| M2 | nr | nr | nr | 6.9 +/− 0.1 | nr* | nr* |
| M3 | nr | nr | nr | 6.7 +/− 0.5 | 8.2 +/− 0.2 | 6.8 +/− 0.7* |
| M4 | nr | nr | nr | 7.4 +/− 0.3 | nr* | nr* |
| M5 | nr | nr | nr | 7.2 +/− 0.2 | 7.5 +/− 0.3 | nr* |
| D3 | 9.7 +/− 0.1 | 7.9 +/− 0.4 | 7.5 +/− 0.3 | 7.6 +/− 0.4 | 6.3 +/− 0.1 | nr |
| Alpha 1A | 7.4 +/− 0.1 | 8.5 +/− 0.1 | 7.4 +/− 0.2 | 7.4 +/− 0.2 | 8.1 +/− 0.1 | 7.3 +/− 0.1 |
| Alpha 2A | nr | 7.7 +/− 0.1 | nr | nr | nr | nr |
| | Inverse Agonist | | | | | |
| Receptor | pEC50 | pEC50 | pEC50 | pEC50 | pEC50 | pEC50 |
| 5-HT2A | 6.8 +/− 0.1 | 9.0 +/− 0.3 | 8.8 +/− 0.3 | 7.8 +/− 0.1 | 8.0 +/− 0.3 | 8.0 +/− 0.3 |
| 5-HT6A | nr | nr | nr | 7.4 +/− 0.2 | 7.0 +/− 0.2 | 6.9 +/− 0.1 |
| 5-HT7A | nr | 9.1 +/− 0.2 | 7.3 +/− 0.1 | nr | 7.4 +/− 0.1 | 7.3 +/− 0.1 |

Competitive antagonism of D₂ receptors, and inverse agonism of 5-HT_{2A} receptors was nearly uniform throughout this class, with typical agents demonstrating low 5HT_{2A}/D₂ ratios, and atypical agents demonstrating high ratios (Meltzer et al 1989 and Weiner et al 2001). Inverse agonism of H₁ receptors was commonly observed, where clozapine and olanzapine displayed particularly high potency (Weiner et al 2001). Many compounds showed antagonist activity at alpha₁-adrenergic receptors, fewer agents exhibited potent 5-HT₆ activity, while many, particularly risperidone, displayed potent inverse agonist activity at 5-HT₇ receptors. Clozapine, olanzapine, and a number of typical agents (e.g. thioridazine, data not shown), were found to possess potent muscarinic receptor antagonist properties. Importantly, no single antagonist activity differentiated clozapine from all other agents.

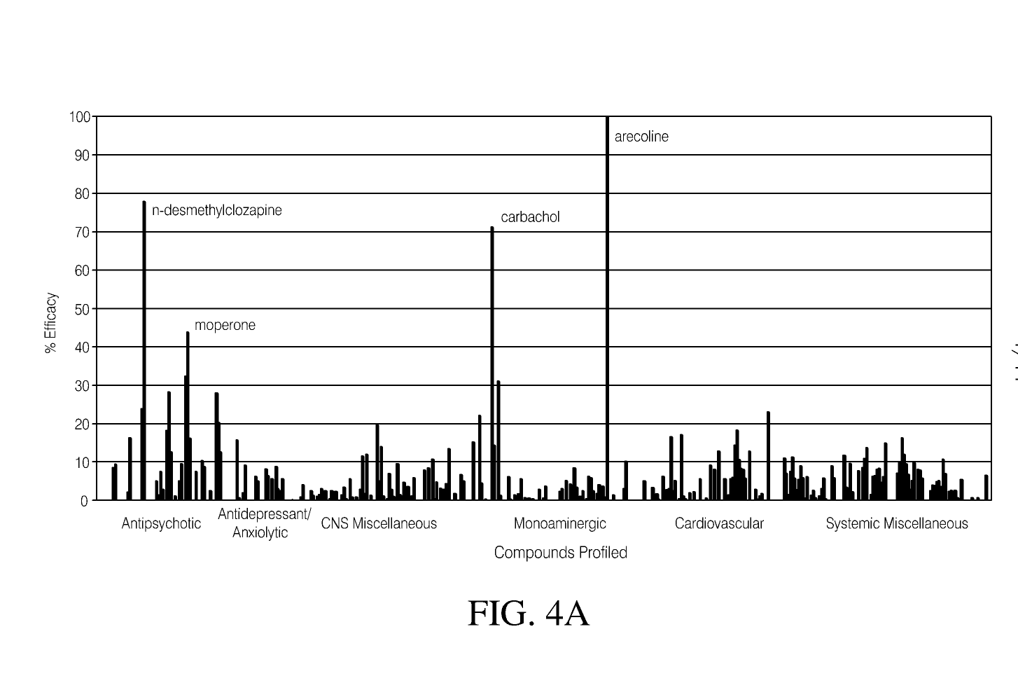

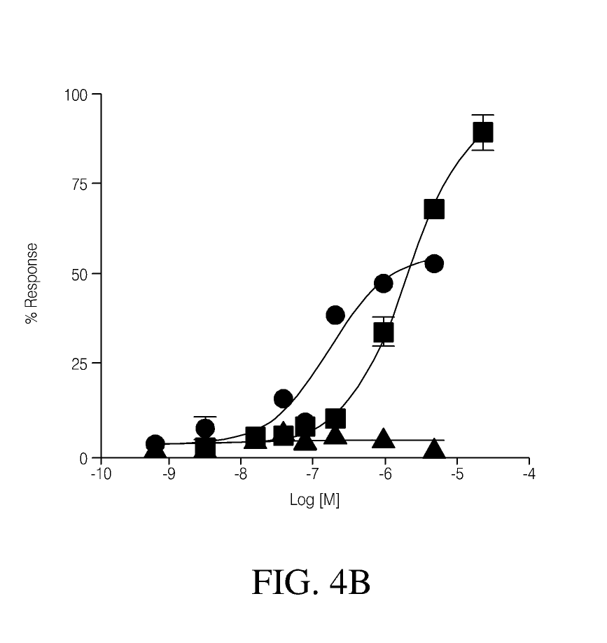

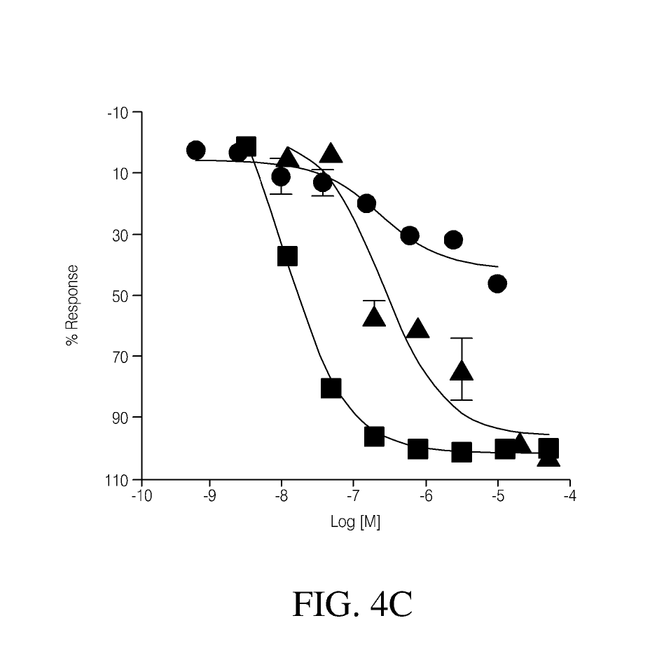

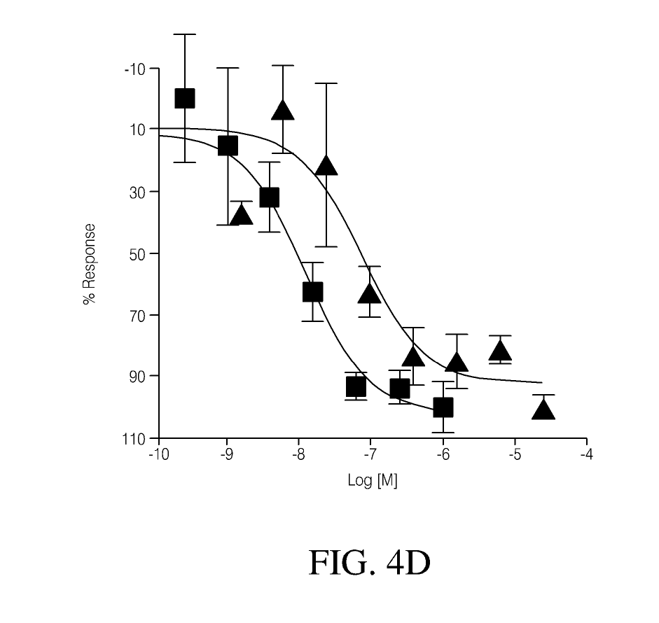

In contrast to the widespread antagonist activity of these compounds, very few agents possessed agonist activity. FIG. 4A reports the results of the functional agonist screen of this compound library at the human M1 muscarinic acetylcholine receptor. Only four compounds, the known muscarinic receptor agonists arecoline and carbachol, moperone and N-desmethylclozapine (NDMC), the major metabolite of clozapine (Gauch and Michaelis 1971), were identified. Moperone displayed only a very low potency (EC₅₀>1-micromolar) interaction. In contrast, NDMC displayed an EC₅₀ of 100 nM with 80% efficacy (relative to carbachol) in this study. This result was further confirmed in a second functional assay, PI hydrolysis. As depicted in FIG. 4B, clozapine displays limited agonist efficacy in this assay, precluding accurate potency determinations, whereas NDMC displayed high potency (93±22 nM, n=3) and greater agonist efficacy (56±8%, n=3) relative to carbachol. In fact, when assayed against carbachol for competitive antagonist activity, clozapine behaved as an antagonist, while NDMC only partially reversed carbachol-induced PI hydrolysis (FIG. 4C), consistent with the lack of an antagonistic response observed when NDMC was tested as a competitive antagonist at M1 receptors in R-SAT (Table 13). Finally, the agonist activity of NDMC was blocked by both atropine and clozapine (FIG. 4D). These results confirm that NDMC is a potent, efficacious, M1 receptor agonist, distinguishing it from the M1 receptor antagonist properties of clozapine.

Having demonstrated the agonist activity of NDMC at human M1 receptors in multiple in vitro functional assays, we then profiled carbachol, clozapine, NDMC, olanzapine, the major olanzapine metabolite N-desmethylolanzapine, and the muscarinic agonist xanomeline (Shannon et al 1994), at all five human muscarinic receptor subtypes using R-SAT (Table 14).

TABLE 14
Muscarinic acetylcholine receptor agonist activity of antipsychotics.
Muscarinic receptor (M1-M5) agonist activity of clozapine, N-desmethylclozapine,
olanzapine, N-desmethylolanzapine, xanomeline, and carbachol was determined using R-SAT
as previously described (Spalding et al 2002). Average efficacy (percentage relative to
carbachol) and potency (pEC₅₀) +/− standard error are reported for 3 or more replicate
determinations. No response denotes the lack of agonist activity at concentrations up to 10-
micromolar.

| | M1 | | M2 | | M3 | |
|---|---|---|---|---|---|---|
| Compound | Efficacy | pEC₅₀ | Efficacy | pEC50 | Efficacy | pEC50 |
| Clozapine | 24 ± 3 | 7.63 ± 0.37 | 65 ± 8 | 6.23 ± 0.14 | No response | |
| N-desmethylclozapine | 72 ± 5 | 7.26 ± 0.07 | 106 ± 19 | 6.47 ± 0.21 | 27 ± 4 | 6.49 ± 0.18 |
| Olanzapine | No response | | No response | | No response | |
| N-desmethylolanzapine | No response | | No response | | No response | |
| Xanomeline | 121 ± 6 | 7.20 ± 0.08 | 106 ± 9 | 6.30 ± 0.23 | 66 ± 6 | 6.63 ± 0.21 |
| Carbachol | 101 ± 2 | 6.11 ± 0.03 | 101 ± 5 | 6.23 ± 0.09 | 102 ± 3 | 6.53 ± 0.04 |

| | |
|---|---|
| M4 | M5 |

| | | | | |
|---|---|---|---|---|
| Compound | Efficacy | pEC₅₀ | Efficacy | pEC50 |

| | | | |
|---|---|---|---|
| Clozapine | 57 ± 5 | 7.35 ± 0.10 | No response |

| | | | | |
|---|---|---|---|---|
| N-desmethylclozapine | 87 ± 8 | 6.87 ± 0.17 | 48 ± 6 | 7.63 ± 0.25 |

| | | |
|---|---|---|
| Olanzapine | No response | No response |
| N-desmethylolanzapine | No response | No response |

| | | | | |
|---|---|---|---|---|
| Xanomeline | 116 ± 9 | 7.46 ± 0.14 | 86 ± 12 | 6.59 ± 0.22 |
| Carbachol | 96 ± 3 | 6.53 ± 0.05 | 105 ± 3 | 6.76 ± 0.12 |

Clozapine was found to be a very weak partial agonist at M1 receptors, a more efficacious agonist at M2 and M4 receptors, and to lack agonist activity at M3 and M5 receptors. NDMC also displayed high potency interactions with all five human muscarinic receptors, but with increased agonist efficacy at M1, M4, and M5 receptors when compared to clozapine (Table 14). In contrast, olanzapine and N-desmethylolanzapine, both structurally related to clozapine and NDMC, lacked agonist activity at human muscarinic receptors. Interestingly, xanomeline displayed a muscarinic receptor profile that is similar to that observed for NDMC, with the notable exception of higher agonist efficacy at M3 receptors. The agonist activities of clozapine, NDMC, and xanomeline at human muscarinic receptor subtypes are unique among all neuropsychiatric agents tested (FIG. 4, and Tables 13 and 14).

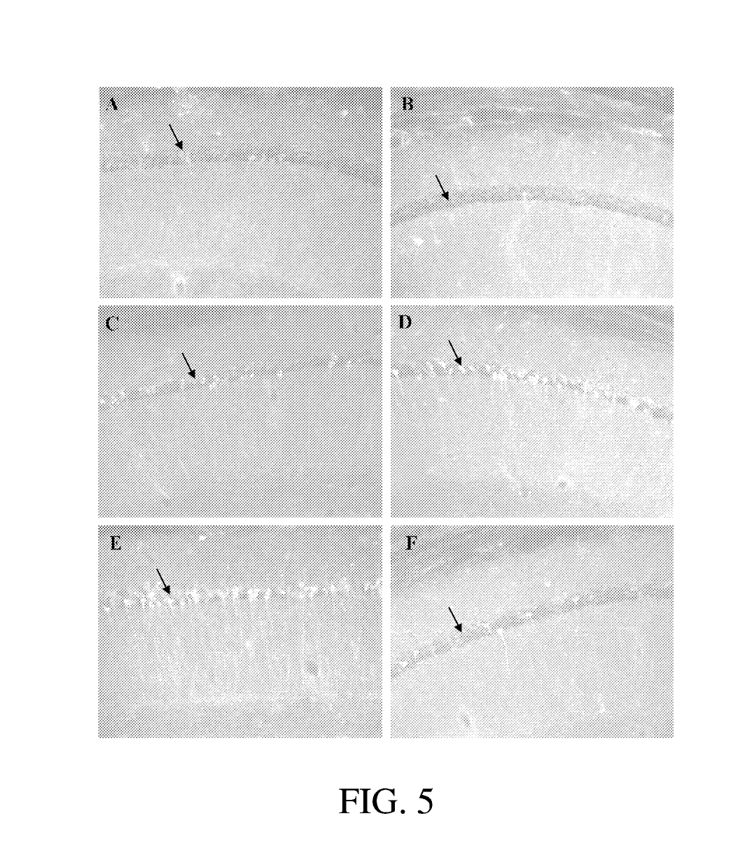

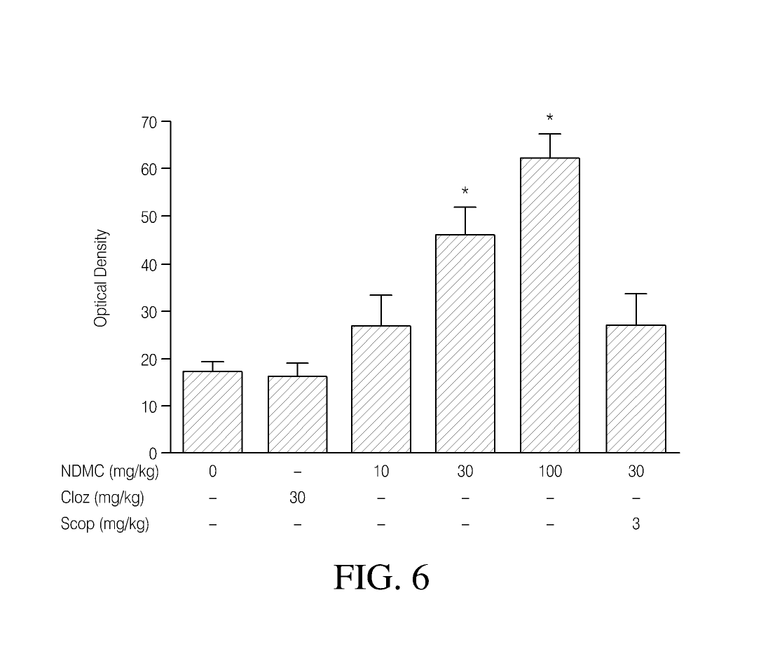

The present inventors discovered that muscarinic receptor agonism, and M1 receptor agonism in particular, of NDMC can be achieved in vivo during pharmacotherapy with clozapine. Clozapine and NDMC were tested for their ability to increase the phosphorylation of mitogen-activated protein kinase (MAP kinase) in the CA1 region of mouse hippocampus, a response that has been shown to reflect M1 receptor activation (Berkeley et al 2001). As depicted in FIG. 5, subcutaneous administration of vehicle (FIG. 5A), clozapine (FIG. 5B), or scopolamine alone (data not shown) fails to stimulate phosphorylation of hippocampal MAP kinase. In contrast, NDMC induced phosphorylation of MAP kinase in hippocampal neurons in a dose dependent manner (FIGS. 5C, 5D, and E), an effect that was blocked by pretreatment with scopolamine (FIG. 5F). Quantification of this effect demonstrates statistically significant M1 receptor activation at NDMC doses of 30 mg/kg and greater (FIG. 6). Clozapine fails to behave as an agonist under these experimental conditions, which likely reflects either insufficient metabolism to NDMC after acute administration in mouse, or direct antagonist effects at the M1 receptor as demonstrated in the in vitro studies. These data confirm that NDMC passes the blood brain barrier and activates hippocampal M1 receptors in vivo.

It has long been appreciated that antagonism of central muscarinic receptors can attenuate the EPS induced by antipsychotics (Miller and Hiley 1974). Initial investigations of the anti-muscarinic properties of antipsychotics defined the high potency of clozapine for these receptors in rodent brain, and elucidated the inverse correlation between muscarinic receptor antagonism and propensity to induce EPS (Snyder et al 1974). Following the elucidation of five muscarinic acetylcholine receptor subtypes (Bonner et al 1987), clozapine was described as a potent competitive antagonist (Bolden et al 1991). Functional studies in various cell lines subsequently documented that clozapine has significant agonist activity at M2 and M4 receptors, and low agonist efficacy at MI receptors (Zorn et al 1994 and Olianas et al 1999), consistent with the results reported herein. In humans, clozapine has two major metabolites, NDMC and clozapine-N-oxide (Gauch and Michaelis 1971). After steady state dosing, NDMC represents a large proportion of total detectable moieties, with concentrations ranging from 20-150% of that observed for clozapine, with mean values of 60-80% (Bondesson and Lindstrom 1988 and Perry et al 1991). That NDMC is an active metabolite is supported by the present data, as well as by prior reports documenting D₁, D₂, and 5-HT_{2C} receptor competitive antagonist activity (Kuoppamaki et al 1993), and a recent report of M1 receptor agonist activity (Sur et al 2003). In contrast, the other major clozapine metabolite, clozapine-N-oxide, displays only very low potency (pKI's<6.0) functional activity at human monoaminergic receptors (data not shown). While varying degrees of brain penetration of NDMC have been reported in rodents (Baldessarini et al 1993 and Weigmann et al 1999), the present results, the observation that systemically administered NDMC activates cFOS expression in rodent brain (Young et al 1998), and the detection of NDMC in human cerebrospinal fluid following parenteral administration of clozapine (Nordin et al 1995), demonstrate that NDMC is brain penetrant and centrally active.

The present inventors have discovered that clozapine, acting through its predominant metabolite NDMC, functions as a direct acting muscarinic receptor agonist in vivo. During pharmacotherapy with clozapine, the agonist actions of NDMC is attenuated by the antagonistic actions of the parent compound. Thus, high NDMC levels, and particularly high NDMC/clozapine ratios, increases agonist efficacy at muscarinic receptors, as predicted by mass action and by agonist/antagonist mixing studies (Brauner-Osborne et al 1996). Clinical data support this notion. Not only does clozapine therapy usually lack the traditional anti-cholinergic side effects of dry mouth, blurred vision, and urinary retention common to classical muscarinic antagonists, it is unique in its ability to frequently produce sialorrhea (Baldessarini and Frankenburg 1991), an effect that can be blocked by the muscarinic antagonist pirenzepine (Fritze and Elliger 1995). Thus, the muscarinic receptor agonist activity of NDMC likely mediates this peripheral effect, while the muscarinic receptor subtype responsible is still unknown, receptor subtypes in addition to the M3 have been implicated (Bymaster et al 2003).

The muscarinic agonist properties of NDMC reported herein underlies some of the unique central effects of treatment with clozapine. Multiple lines of evidence support a pro-cognitive effect of potentiating central cholinergic neurotransmission, including the clinical effects of acetylcholinesterase inhibitors and direct acting muscarinic receptor agonists (Davis et al 1993). High dose clozapine therapy in treatment refractory schizophrenics may actually serve to raise brain levels of NDMC to achieve central muscarinic receptor agonist activity, particularly MI receptor stimulation, rather than recruiting additional lower potency receptor interactions that clozapine and NDMC possess (Table 13). Thus, NDMC/clozapine ratios are a better predictor of therapeutic response to clozapine, particularly for cognition, than absolute clozapine levels.

The data on clozapine and NDMC plasma levels and clinical response that were prospectively gathered as part of two clinical trials which included 59 neuroleptic resistant patients (Hasegawa et al 1993), and 33 neuroleptic responsive patients (Lee et al 1999) with schizophrenia were re-analyzed. Patients were classified as treatment resistant or not by standard criteria (Kane et al 1988), and clinical ratings and neuropsychological test scores were obtained by trained raters who were blinded to plasma drug levels. The mean daily dosages of clozapine, as well as clozapine and NDMC serum levels, and NDMC/Clozapine ratios after 6 weeks and 6 months of treatment are reported in Table 15A.

TABLE 15
Serum N-desmethylclozapine levels and clinical response in schizophrenia.
Table 15A

| | All TR | | Non- | |
|---|---|---|---|---|
| | Subjects | Responders | Responders | |
| Drug Measure | (59) | (26) | (25) | P* |
| Dose (mg/day) | 468 +/− 190 | 485 +/− 205 | 433 +/− 178 | NS |
| NDMC Level | 260 +/− 203 | 308 +/− 243 | 171 +/− 123 | 0.01 |
| (ng/ml) | | | | |
| Clozapine | 393 +/− 301 | 453 +/− 328 | 268 +/− 207 | 0.02 |
| Level (ng/ml) | | | | |
| NDMC/ | 0.75 +/− 0.36 | 0.70 +/− 0.22 | 0.81 +/− 0.48 | NS |
| Clozapine | | | | |

| | All Subjects at 6 | All Subjects at 6 Months |
|---|---|---|
| Drug Measure | Weeks (86) | (92) |
| Dose (mg/day) | 369 +/− 169 | 417 +/− 197 |
| NDMC Level (ng/ml) | 194 +/− 136 | 235 +/− 190 |
| Clozapine Level (ng/ml) | 287 +/− 190 | 365 +/− 285 |
| NDMC/Clozapine | 0.83 +/− 1.08 | 0.71 +/− 0.30 |

Table 15B

| Clinical Measure | Beta | F | P | r²** | df |
|---|---|---|---|---|---|
| Dependent Variable: 6 Weeks | | | | | |
| BPRS-Withdrawal/Retardation | −0.52 | 3.73 | 0.06 | 0.32 | 3.73 |
| SANS Attentional Impairment | −0.28 | 5.65 | 0.02 | 0.26 | 3.65 |
| SAPS Global Delusions | −1.00 | 3.87 | 0.05 | 0.60 | 3.55 |
| Quality of Life Scale: Total | 17.50 | 5.20 | 0.03 | 0.50 | 2.40 |
| Quality of Life Scale: Objects and | 2.91 | 7.10 | 0.01 | 0.43 | 2.40 |
| Activities | | | | | |
| Quality of Life Scale: Instrumental | 13.80 | 14.84 | 0.01 | 0.54 | 2.39 |
| Role | | | | | |
| WISC-R Maze | 2.27 | 4.10 | 0.05 | 0.75 | 4.33 |
| Dependent Variable: 6 Months | | | | | |
| Petersen's Consonant Trigram Test | 7.45 | 6.75 | 0.01 | 0.47 | 4.47 |
| WISC-Categories Formed | 1.35 | 3.67 | 0.06 | 0.47 | 3.48 |
| Statistical analysis of the correlation between clinical outcome and serum levels of clozapine and N-desmethylclozapine (NDMC) for a cohort of 92 clozapine treated schizophrenics are reported. Table 15A reports the clozapine dose, clozapine level, NDMC levels, and NDMC/clozapine ratios for all treatment resistant (TR) subjects, responders, non-responders, and all subjects at 6 weeks and 6 months. P* reports statistically significant differences between responders and | | | | | |
| # non-responders. Table 15B report treatment, including quality of life, negative symptoms, and cognition, analyzed by multiple linear regression. R²** refers to the model applied. Abbreviations used include: NS-not significant, BPRS-Brief Psychiatric Rating Scale, SANS-Scale for the Assessment of Negative Symptoms, SAPS-Scale for the Assessment of Positive Symptoms, WISC-Wisconsin Card Sorting Test. | | | | | |

Both time points were analyzed because improvement in psychopathology and cognition with clozapine may take six months or longer (Hagger et al 1993). Thirteen of the 92 patients (14.1%) had NDMC/clozapine ratios >/=1. Of these thirteen patients, the highest ratio was 1.77 and the median was 1.05. The Spearman rank order correlation between clozapine and NDMC levels was 0.82 and 0.89 at 6 weeks and 6 months, respectively (P=0.0001). The correlation between NDMC/clozapine ratios at 6 weeks and 6 months was 0.92 (P=0.0001), indicating remarkable stability of NDMC/clozapine ratios within subjects. Importantly, dose and NDMC/clozapine ratios were not significantly correlated at either time point (rho<0. 10) in neither the neuroleptic-resistant nor neuroleptic-responsive patients.

Stepwise multiple-regression were utilized to determine the best predictors of outcome from each of these measures, including baseline levels of the dependent measure, dose, age, and gender, since all have been shown to significantly predict response to clozapine (Table 15B).

In all the models tested, baseline levels of the dependent measure predicted the largest share of the variance in the model. The NDMC/clozapine ratio was the next most frequent predictor of response; the ratio significantly predicted response in 8/24 (33.3%) of the models, all in the expected direction: the higher the ratio, the better the outcome. This result contrasts with the lack of predictive power of clozapine levels alone, NDMC levels alone, or their sum. The exception was that higher NDMC levels alone predicted greater improvement in two subscales of the Quality of Life scale (Heinrichs et al 1984) (data not shown). As shown in Table 15B, higher NDMC/clozapine ratio predicted improvement in multiple measures of cognition, as well as the Scale for the Assessment of Negative Symptoms-Attention subscale, which has been suggested to be more related to cognition than negative symptoms. The ratio also predicted improvement in Quality of Life-total score, including the Instrumental Role Function factor, which has been shown to be dependent upon cognitive status (Green 1996), and negative symptoms, which have been found to correlate with cognition. The ratio also predicted improvement in delusions, but not hallucinations, with clozapine treatment. Dose did not contribute to the prediction of any of the models in Table 15B. Dose is significantly correlated with plasma levels of clozapine and NDMC (P=0.01-0.001) but not, as noted above, with the NDMC/clozapine ratio. This provides further evidence that the absolute levels of clozapine and NDMC, while important in identifying responders and non-responders (Fabrazzo et al 2002) are not as important as their ratio when baseline levels of the dependent measure are included in the model. Although additional analyses in larger cohorts are necessary, this analysis, as well as recent reports (Frazier et al 2003 and Mauri et al 2003) all suggest that the NDMC/clozapine ratio is a better predictor of clinical response to clozapine than clozapine levels alone, and support the hypothesis that NDMC is a critical mediator of clozapine action.

The muscarinic receptor agonist properties of NDMC also contribute to the efficacy of clozapine therapy against positive symptoms. Not only did high NDMC/clozapine ratios predict response to delusions as noted above, but additional support comes from the observation that there are several similarities between the central effects of muscarinic receptor agonists and dopamine D₂ receptor antagonists (Pfeiffer and Jenney 1957 and Mirza et al 2003). For example, behavioral pharmacological experiments with mice harboring targeted deletions of each of the five muscarinic receptor subtypes have shown that the M1 receptors plays a central role in DA-mediated behaviors (Gerber et al 2001). In addition, xanomeline (which displays some selectivity for M1 and M4 receptors) inhibits amphetamine-induced locomotion (Shannon et al 2000). Clinically, xanomeline was found to diminish hallucinosis and aggression in Alzheimer's Disease patients (Bodick et al 1997), and has been shown to display activity against both positive and negative symptoms in a recent, small, Phase 2 study in schizophrenia (Schekhar et al, unpublished data).

The central dopaminergic and muscarinic cholinergic systems are well known to be functionally interrelated (Miller and Hiley 1974). The muscarinic antagonist properties of clozapine are thought to contribute to its low propensity to cause EPS, yet the anti-EPS effects of clozapine are more robust than those obtained by the adjunctive use of anticholinergics agents like trihexyphenidyl, and some EPS producing antipsychotics, e.g. thioridazine, also possess potent muscarinic receptor antagonist properties. These observations suggest that although antagonism of central muscarinic receptors can confer anti-EPS effects, cholinergic modulation of the motoric effects of D₂ receptor blockade are more complex than previously appreciated. Present data show that agonism, not antagonism, of certain muscarinic receptor subtypes expressed within critical basal ganglia structures (Weiner et al 1990), are a more efficacious mechanism to lessen these adverse motor effects. Further, the widespread use of adjunctive anticholinergics should be reevaluated in light of the present data on the pro-cognitive benefits conferred by the central muscarinic receptor agonist properties of NDMC.

In summary, functional characterization of therapeutically useful neuropsychiatric drugs has revealed the potent, efficacious, muscarinic receptor agonist activity of NDMC. This activity was found to be unique among neuropsychiatric agents as a class. It is demonstrated that NDMC can cross the blood brain barrier and function as an M1 receptor agonist in vivo. Consideration of the contribution of NDMC to improvement in cognition and quality of life in clozapine treated patients shows that NDMC mediates clinically relevant aspects of treatment response that differentiate clozapine from other agents used to treat schizophrenia. These findings show that muscarinic receptor agonism mediates the unique clinical properties of clozapine, and that M1 muscarinic receptor agonists (Spalding et al 2002), including NDMC itself, may be efficacious atypical antipsychotic agents.

### Example 14

### Net Agonism in N-desmethylclozapine/Clozapine Mixtures

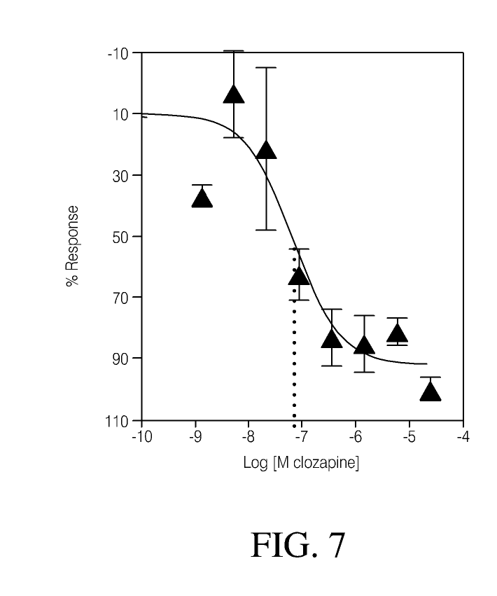

The effect of mixtures of clozapine and N-desmethylclozapine was evaluated using an R-SAT assay as described above. 150 nM of N-desmethylclozapine was provided with varying concentrations of clozapine. FIG. 7 depicts the results of the R-SAT assay as a function of clozapine concentration. As indicated by the dotted line in FIG. 7, net agonistic activity was observed for clozapine concentrations of about 100 nM and below. Thus, ratios of NDMC to clozapine of about 1.5 and greater provide a net agonistic effect.

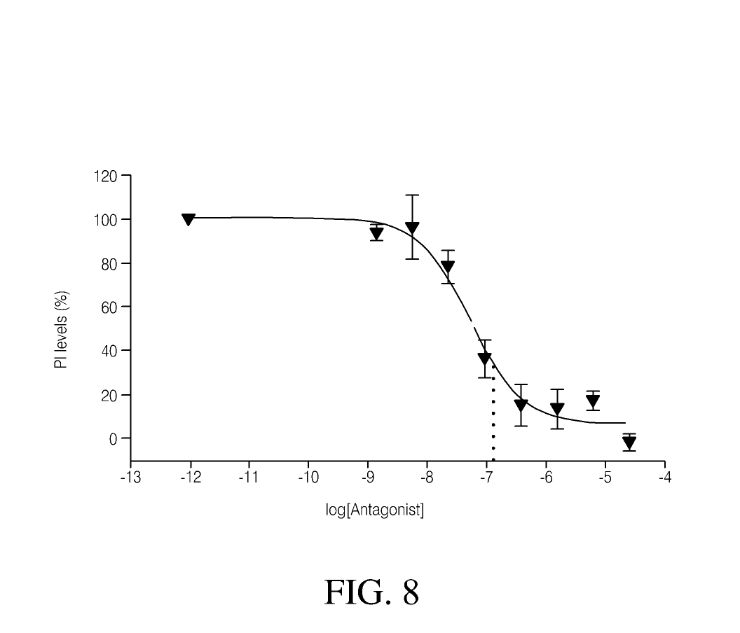

The results of the R-SAT assay were confirmed using a PI hydrolysis assay as described above. 150 nM of N-desmethylclozapine was again provided with varying concentrations of clozapine. FIG. 8 depicts the results of the assay as a function of clozapine concentration. The dotted line in FIG. 8 indicates the maximum concentration of clozapine for which a net agonistic effect is observed. Similar to the results of the R-SAT assay, net agonistic activity was observed for clozapine concentrations of about 100 nM and below, thus confirming that a ratio of NDMC to clozapine of about 1.5 and greater provide a net agonistic effect.

### Example 15

### Administration of Single Doses of NDMC to Schizophrenic Patients

A single-center, in-patient, randomized, double blind, placebo controlled, single dose study is conducted on two sequential group of patients. Two different groups of 6 patients each are enrolled. Each patient in the first group of patients receives single doses of placebo, 25 mg of NDMC, and 50 mg of NDMC sequentially in random order. Each patient in the second group of patients receives single doses of placebo, 75 mg of NDMC, and 100 mg of NDMC sequentially in random order. The NDMC and placebo is administered orally as a powder in a gelatin capsule. Male or female patients, 20 to 50 years of age, with a history of schizophrenia or schizoaffective disorder, who are otherwise in good health are selected for the study. The patients are not experiencing acute exacerbation of severe psychosis, defined as a Positive and Negative Syndrome Scale (PANSS) score greater than 75.

Patients are withdrawn from all centrally active medications during a lead-in period of 4-7 days prior to study start on Study Day -1. On Study Day-1, patients are randomized to a schedule of NDMC:placebo in a 2:1 manner. On Study Day 1, patients receive study drug or placebo, orally, in the morning, and serial blood samples are collected up to 24 h after dose administration. Patients are monitored for 8 hr post-dose by continuous lead II ECG monitoring. Clinical evaluation, clinical rating scales, and frequent assessments of safety including vital signs, ECGs, clinical labs, and adverse event recording are performed periodically throughout Study Day. No study drug is given on Study Days 2 and 3. On Study Day 4, subjects once again receive study drug or placebo, orally, in the morning, and serial blood samples are collected up to 24 h after dose administration. Patients are monitored for 8 h post-dose by continuous lead II ECG monitoring. Clinical evaluation, clinical rating scales, and frequent assessments of safety including vital signs, ECGs, clinical labs, and adverse event recording are performed periodically throughout the Study Day 4. No study drug or placebo is given on Study Days 5 and 6. On Study Day 8, patients receive study drug or placebo, orally, in the morning, and serial blood samples are collected up to 24 h after dose administration. Patients are monitored for 8 h post-dose by continuous lead II ECG monitoring. Clinical evaluation, clinical rating scales, and frequent assessments of safety including vital signs, ECGs, clinical labs, and adverse event recording are performed periodically throughout the Study Day 8. A final End of Study evaluation is performed 3-5 days following Study Day 8 and a clinical evaluation, administration of clinical rating scales, and a safety assessment are performed.

An interim analysis of the safety variables and pharmacokinetic data obtained from Group 1 is conducted after the End of Study evaluation and before the randomization of Group 2. Safety variables are reviewed by the PI in order to determine the doses to be administered in Group 2. If NDMC administration during Group 1 is deemed safe the second patient cohort is screened, randomized, and enrolled. If the doses of NDMC in Group 2 are greater than those administered in Group 1, then, during the lead-in period, a pre-conditioning dose of 25 mg of NDMC is given to each subject. This test dose is used to identify any patient who may be particularly sensitive to higher doses of NDMC, and is administered at least 3 days prior to Day -1. Study related procedures for Group 2 is identical to those of Group 1, with the exception of the NDMC dose.

Pharmacokinetic Analysis

Plasma samples are analyzed for concentrations of NDMC. Pharmacokinetic parameters are calculated including Cₘₐₓ (maximum plasma concentration), tₘₐₓ (time to maximum plasma concentration), AUC_{0-z} (area under the plasma concentration time curve from time zero to the last quantifiable timepoint, calculated by linear-log trapezoidal summation), AUC_{0-∞} (area under the plasma concentration time curve from time zero to infinity, calculated by linear-log trapezoidal summation and extrapolated to infinity by addition of the last quantifiable plasma concentration divided by the elimination rate constant λ_{Z}), λ_{Z} (elimination rate constant, determined by linear regression of the terminal points of the log-linear plasma concentration-time curve), t_{1/2} (terminal half-life, determined as ln(2)/λ_{Z}), and CLₚₒ (apparent oral clearance, calculated by Dose/AUC(0−∞)).

Tolerability

Tolerability of NDMS is determined by measuring extrapyramidal (EPS) motor effect using the Simpson and Angus Scale (SAS) and the Barnes Akathisia Scale (BAS). These scales are administered at baseline (Study Day -1), 3-5 hours after drug administration on Study Days 1, 4, and 8, and at the End of Study evaluation.

Antipsychotic Efficacy

Antipsychotic efficacy is measured using the PANSS and the Clinical Global Impression Scale-Schisophrenia (CGI-S) measures. These scales are administred at baseline (Study Day -1), on Study Days 1, 4, and 8, and at the End of Study evaluation.

Safety

Safety is evaluated by measuring vital signs including 3-positional blood pressure and pulse rate (5 minute supine, 1 minute sitting, 3 minutes standing), respiratory rate, and oral temperature except during screening and post-study procedures.

12-lead ECGs are recorded and standard electrocardiogram parameters including QRS, PR, QT, and QTc intervals are measured. In addition, continuous lead-II ECG monitoring is performed for the first 8 hours of Day 1, 4, and 8 following each NDMC or placebo dose administration.

A neurological screen is conducted by the clinically responsible physician at the clinic. The neurological screen consists of a qualitative assessment of muscle tone in the extremities, the presence of tremors, fasiculations, and nystagmus, and various tests of cerebellar coordination (finger nose test, dysdiadochokinesia, heel-shin test, and gait).

Clinical laboratories are measured after at least an 8-hour fast on Study Days 1, 4, 8, and the End of Study evaluation and include the following:

Erythrocytes: RBC count, hematocrit, hemoglobin, mean corpuscular hemoglobin (MCH), mean corpuscular hemoglobin concentration (MCHC), mean corpuscular volume (MCV), RDW, and reticulocyte count.

Leukocytes: WBC count and differential (basophils, eosinophils, lymphocytes, monocytes, and neutrophils) reported as absolute values.

Coagulation: platelet count, PT as INR, and aPTT.

Liver: alkaline phosphatase, ALT (serum glutamic-pyruvic transaminase [SGPT]), AST (serum glutamic-oxaloacetic transaminase [SGOT]), bilirubin (total, direct, indirect), gamma-glutamyl transferase (GGTP), creatine phosphokinase (CPK) and LDH.

Renal: blood urea nitrogen (BUN), creatinine, and uric acid.

Electrolytes: carbon dioxide, chloride, magnesium, potassium, and sodium.

General: albumin, calcium, glucose (fasting) phosphate, and protein (total).

Endocrine: prolactin.

Lipids: cholesterol (total), HDL cholesterol, LDL cholesterol, and triglycerides.

Macroscopic urinalysis: pH, specific gravity, glucose, ketones, leukocyte esterase, nitrites, occult blood, and protein.

Microscopic urinalysis: RBC/high powered field, WBC/high powered field, bacteria, castes, epithelial cells, mucous threads and crystals.

### Example 16

### Administration of Multiple Doses of NDMC to Schizophrenic Patients

A single-center, in-patient, randomized, double blind, placebo controlled, multiple dose study is conducted on two sequential groups. Twelve patients, in two different groups of six patients each are enrolled. Each patient receives either placebo or NDMC daily for five days. The NDMC and placebo is administered orally as a powder in a gelatin capsule. Male or female patients, 20 to 50 years of age, with a history of schizophrenia or schizoaffective disorder, who are otherwise in good health are selected for the study. The patients are not experiencing acute exacerbation of severe psychosis, defined as a Positive and Negative Syndrome Scale (PANSS) score greater than 75.

Patients are withdrawn from all centrally active medications during the lead-in period of 4-7 days prior to study start on Study Day -1. If the safety profile of NDMC as determined by the single-dose study of Example 14 suggests that a gradual dose escalation is warranted, and if the pharmacokinetics properties of NDMC demonstrate that the t_{1/2} is less than 8 hr. then patients enrolled in the study receive pre-conditioning doses of NDMC prior to Study Day -1. If indicated, subjects receive, during the lead-in portion of the study, a single dose of NDMC that is 25% of the planned dose, followed by a second dose the following day which is 50% of the planned dose. If these doses are deemed safe, then subjects are randomized on Study Day -1 to either NDMC or placebo. The dosages and frequency of administration (QD or BID) of NDMC are determined based on the safety and pharmacokinetics observed during the single dose safety study of Example 14.

On Study Day 1, patients receive study drug or placebo, orally, and serial blood samples are collected up to 24 hours after dose administration. Patients are monitored for 8 hr post-dose by continuous lead II ECG monitoring. Clinical evaluation, clinical rating scales, and frequent assessments of safety including vital signs, ECGs, clinical labs, and adverse event recording are performed periodically throughout Study Day 1.

Patients receive study drug or placebo daily for the next four days. On Study Days 2, 3, and 4, pre-dose serum sampling for pharmacokinetic analysis are obtained, and patients are monitored for 8 hr post-dose by continuous lead II ECG monitoring. Clinical evaluation, clinical rating scales, and frequent assessments of safety including vital signs, ECGs, clinical labs, and adverse event recording are performed periodically throughout Study Days 2-4.

On Study Day 5, pre-dose serum sampling as well as serial blood samples collected up to 24 hours after study drug or placebo administration are obtained for pharmacokinetic analysis, and patients are monitored for 8 hr post-dose by continuous lead II ECG monitoring. Clinical evaluation, clinical rating scales, and frequent assessments of safety including vital signs, ECGs, clinical labs, and adverse event recording are performed periodically throughout Study Day 5.

A final End of Study evaluation is conducted 5-7 days after the cessation of active dosing on Study Day 5. A safety assessment is performed during this clinical evaluation, including vital signs, ECG, clinical labs, NDMC serum determination, and adverse event recording. All patients are followed clinically, as in-patients, for as long as is indicated following the cessation of active dosing of NDMC.

An interim analysis of the safety variables and pharmacokinetic data from Group 1 is conducted after the End of Study evaluation, and before the randomization of Group 2. Safety variables are reviewed by the PI in order to determine the doses to be administered in Group 2. If NDMC administration during Group 1 is deemed safe, the second patient cohort is screened, randomized, and enrolled. Study related procedures for Group 2 are identical to those of Group 1, with the exception of NDMC dose and/or frequency of administration.

Pharmacokinetic Analysis

Plasma samples are analyzed for concentrations of NDMC. Pharmacokinetic parameters are calculated are calculated following the single dose administration on Day 1 including Cmax (maximum plasma concentration), tₘₐₓ (time to maximum plasma concentration), AUC_{0-z} (area under the plasma concentration time curve from time zero to the last quantifiable timepoint, calculated by linear-log trapezoidal summation), AUC_{0-∞} (area under the plasma concentration time curve from time zero to infinity, calculated by linear-log trapezoidal summation and extrapolated to infinity by addition of the last quantifiable plasma concentration divided by the elimination rate constant λ_{Z}), λ_{Z} (elimination rate constant, determined by linear regression of the terminal points of the log-linear plasma concentration-time curve), t_{1/2} (terminal half-life, determined as ln(2)/λ_{Z}), and CLₚₒ (apparent oral clearance, calculated by Dose/AUC(0−∞)).

Pharmacokinetic parameters are also calculated following the last dose on Day 5 including C_{max,ss} (maximum steady-state plasma concentration), C_{min,ss} (minimum steady-state plasma concentration), C_{avg,ss} (average steady-state plasma concentration calculated as AUC(0−τ)ₛₛ divided by the dosing interval τ), t_{max,ss} (time to maximum steady-state plasma concentration), t_{mis,ss} (time to minimum steady-state plasma concentration), AUC_{0-z} (area under the plasma concentration time curve from time zero to the last quantifiable timepoint, calculated by linear-log trapezoidal summation), AUC_{0-τss} (area under the plasma concentration time curve from time zero to the end of the steady-state dosing interval, calculated by linear-log trapezoidal summation), λ_{Z,ss} (steady-state elimination rate constant, determined by linear regression of the terminal points of the log-linear plasma concentration-time curve), t_{1/2,ss} (steady-state terminal half-life, determined as ln(2)/λ_{Z,ss}), and CL_{po,ss} (apparent oral clearance, calculated by Dose/AUC(0−τ)ₛₛ).

Tolerability

Tolerability of NDMS is determined by measuring extrapyramidal (EPS) motor effect using the Simpson and Angus Scale (SAS) and the Barnes Akathisia Scale (BAS). These scales are administered at baseline (Study Day -1), 6 hours after drug administration on Study Days 1-5, and at the End of Study evaluation.

Antipsychotic Efficacy

Antipsychotic efficacy is measured using the PANSS and the Clinical Global Impression Scale-Schisophrenia (CGI-S) measures. These scales are administred at baseline (Study Day -1), on Study Days 1, 5, and at the End of Study evaluation.

Safety

Safety is evaluated by measuring vital signs including 3-positional blood pressure and pulse rate (5 minute supine, 1 minute sitting, 3 minutes standing), respiratory rate, and oral temperature except during screening and post-study procedures.

12-lead ECGs are recorded and standard electrocardiogram parameters including QRS, PR, QT, and QTc intervals are measured. In addition, continuous lead-II ECG monitoring is performed for the first 8 hours of Days 1-5 following each NDMC or placebo dose administration.

A neurological screen is conducted by the clinically responsible physician at the clinic. The neurological screen consists of a qualitative assessment of muscle tone in the extremities, the presence of tremors, fasiculations, and nystagmus, and various tests of cerebellar coordination (finger nose test, dysdiadochokinesia, heel-shin test, and gait).

Clinical laboratories are measured after at least an 8-hour fast and include the following:

Erythrocytes: RBC count, hematocrit, hemoglobin, mean corpuscular hemoglobin (MCH), mean corpuscular hemoglobin concentration (MCHC), mean corpuscular volume (MCV), RDW, and reticulocyte count.

Leukocytes: WBC count and differential (basophils, eosinophils, lymphocytes, monocytes, and neutrophils) reported as absolute values.

Coagulation: platelet count, PT as INR, and aPTT.

Liver: alkaline phosphatase, ALT (serum glutamic-pyruvic transaminase [SGPT]), AST (serum glutamic-oxaloacetic transaminase [SGOT]), bilirubin (total, direct, indirect), gamma-glutamyl transferase (GGTP), creatine phosphokinase (CPK) and LDH.

Renal: blood urea nitrogen (BUN), creatinine, and uric acid.

Electrolytes: carbon dioxide, chloride, magnesium, potassium, and sodium.

General: albumin, calcium, glucose (fasting) phosphate, and protein (total).

Endocrine: prolactin.

Lipids: cholesterol (total), HDL cholesterol, LDL cholesterol, and triglycerides.

Macroscopic urinalysis: pH, specific gravity, glucose, ketones, leukocyte esterase, nitrites, occult blood, and protein.

Microscopic urinalysis: RBC/high powered field, WBC/high powered field, bacteria, castes, epithelial cells, mucous threads and crystals.

Literature Cited

Each of the following references is incorporated by reference herein in its entirety, including any drawings.

The following references are incorporated herein by reference in their entireties, including any drawings.

Eglen, R., M., Choppin, A., and Watson, N., (2001) Therapeutic opportunities from muscarinic receptor research. Trends Pharmacol. Sci. 22(8): 409-414.

Brown, J., H., and Taylor, P., (1996) Muscarinic receptor agonists and antagonists, in The pharmacological basis of therapeutics. Hardiman, J., G., and Limbird, L., E., editors, Mcgraw-Hill, New York, pp. 141-161.

Moroi, S., E., and Lichter, P., R. (1996) Ocular pharmacology, in The pharmacological basis of therapeutics. Hardiman, J., G., and Limbird, L., E., editors, Mcgraw-Hill, New York, pp. 1619-1647.

Davis, R E; Doyle, P D; Carroll, R T; Emmerling, M R; Jaen, J. Cholinergic therapies for Alzheimer's disease: Palliative or disease altering? Arzneimittel-Forschung,.45, 425-431, 1995.

Shekhar, A., Potter, W., Z., Lienemann, J., et. al. (2001) Efficacy of xanomeline, a selective muscarinic agonist, in treating schizophrenia: a double blind placebo controlled study. ACNP abstracts 135: 173.

Rodriquez, M. A., Whipple, B., Ocampo, G., et. al. (2002) Muscarinic agonists in neuropathic and nociceptive pain assays in rats. International Association for the Study of Pain's 10^{th} World Congress, 1160-P76: 388.

Baldessarini, R., J., and Frankenburg, F., R. (1991) Clozapine. A novel antipsychotic agent. New. Engl. J. Med., 324(11): 746-754.

Jann, M., W., Grimsley, S., R., Gray, E., C., and Chang, W. (1993) Pharmacokinetic and pharmacodynamics of clozapine. Clin. Pharmacokinet. 24(2): 161-176.

Centorrino, F., Baldessarini, R., J., Kando, J., C., et. al. (1994) Clozapine and metabolites: concentrations in serum and clinical findings during treatment of chronically psychotic patients. J. Clin. Psychopharmacol. 14: 119-125.

Hunziker F. Fisher, E., and Scmutz, J. (1967) 11-amino-5H-dibenzo[b,e]-1,4-diazepine. Mitteilung uber siebenglienrige Heterocyclen. Helv. Chim. Acta, 50:1588-1599.

Jensen, A., A., Spalding, T., A., Burstein E., S., et. al. (2000) Functional importance of the Ala(116)-Pro(136) region in the calcium-sensing receptor. Constitutive activity and inverse agonism in a family C G-protein-coupled receptor. J Biol Chem. 275(38): 29547-55.

Baldessarini R J, Frankenburg F R (1991) Clozapine: a novel antipsychotic agent. N Engl J Med 324:746-754.

Baldessarini R J, Centorrino F, Flood J G, Volpicelli S A, Huston-Lyons D, Cohen B M (1993) Tissue concentrations of clozapine and its metabolites in the rat. Neuropsychopharmacology 9:117-124.

Berkeley J L, Gomeza J, Wess J, Hamilton S E, Nathanson N M, Levey A I (2001) M1 muscarinic acetylcholine receptors activate extracellular signal-regulated kinase in CA1 pyramidal neurons in mouse hippocampal slices. Mol Cell Neurosci 18:512-524.

Bodick N C, Offen W W, Levey A I, Cutler N R, Gauthier S G, Satlin A, Shannon B E, Tollefson G D, Rasmussen K, Bymaster F P, Hurley D J, Potter W Z, Paul S M (1997) Effects of xanomeline, a selective muscarinic receptor agonist, on cognitive function and behavioral symptoms in Alzheimer disease. Arch Neurol 54:465-473.

Bolden C, Cusack B, Richelson E (1991) Clozapine is a potent and selective muscarinic antagonist at the five cloned human muscarinic acetylcholine receptors expressed in CHO-K1 cells. Eur J Pharmacol 192:205-206.

Bondesson U, Lindstrom L H (1988) Determination of clozapine and its N-demethylated metabolite in plasma by use of gas chromatography-mass spectrometry with single ion detection. Psychopharmacology 95:472-475.

Bonner T I, Buckley N J, Young A C, Brann M R (1987) Identification of a family of muscarinic acetylcholine receptor genes. Science 237:527-532.

Brauner-Osborne H, Ebert B, Brann M R, Falch E, Krogsgaard-Larsen P (1996) Functional partial agonism at cloned human muscarinic acetylcholine receptors. Eur J Pharmacol 313:145-150.

Bymaster F B, Carter P A, Yamada M, Gomeza J, Wess J, Hamilton S, Nathanson N M, McKinzie D L, Felder C C (2003) Role of specific muscarinic receptor subtypes in cholinergic parasympathomimetic responses, in vivo phosphoinositide hydrolysis, and pilocarpine-induced seizure activity. Eur J Neurosci 17:1403-1410.

Carlsson A (1978) Antipsychotic drugs, neurotransmitters, and schizophrenia. Am J Psychiatry 135(2):165-173.

Davis R E, Emmerling M R, Jaen J C, Moos W H, Spiegel K (1993) Therapeutic intervention in dementia. Crit Rev Neurobiol 7:41-83.

Fabrazzo M, La Pia S, Monteleone P, Esposito G, Pinto A, De Simone L, Bencivenga R, Maj M (2002) Is time course of clozapine response correlated to the time course of plasma clozapine levels? A one-year prospective study in drug-resistant patients with schizophrenia. Neuropsychopharmacology 27:1050-1055.

Frazier J A, Glassner Cohen L, Jacobsen L, Grothe D, Flood J, Baldessarini R J, Piscitelli S, Kim G S, Rapoport J L (2003) Clozapine pharmacokinetics in children and adolescents with childhood-onset schizophrenia. J Clin Psychopharmacol 23(1):87-91.

Fritze J, Elliger T (1995) Pirenzepine for clozapine-induced hypersalivation. Lancet 346:1034.

Gauch R, MichaelisW (1971) The metabolism of 8-chloro-11-(4-methyl-1-piperazinyl)-5H-dibenzo[b,e][1,4]diazepine(clozapine) in mice, dogs, and human subjects. Farmaco 26:667-681.

Gerber D J, Sotnikova T D, Gainetdinov R R, Huang S Y, Caron M G, Tonegawa S (2001) Hyperactivity, elevated dopaminergic transmission, and response to amphetamine in M1 muscarinic acetylcholine receptor-deficient mice. Proc Natl Acad Sci USA 98(26):15312-15371.

Green M F (1996) What are the functional consequences of neurocognitive deficits in schizophrenia? Am J Psychiatry 153:321-330.

Hagger C, Buckley P, Kenny J T, Friedman L, Ubogy D, Meltzer H Y (1993) Improvement in cognitive functions and psychiatric symptoms in treatment-refractory schizophrenic patients receiving clozapine. Biol Pyschiatry 34:702-712.

Hasegawa M, Gutierrez-Esteinou R, Way L, Meltzer H Y (1993) Relationship between clinical efficacy and clozapine concentrations in plasma in schizophrenia: effect of smoking. J Clin Psychopharmacol 13:383-390.

Heinrichs D W, Hanlon T E, Carpenter W T (1984) The Quality of Life Scale: an instrument for rating the schizophrenia deficit syndrome. Schizophr Bull 10:388-398.

Kane J, Honigfeld G, Singer J, Meltzer H, Clozaril Collaborative Study Group (1988) Clozapine for the treatment-resistant schizophrenic. Arch Gen Psychiatry 45:789-796.

Kuoppamaki M, Syvalahti E, Hietala J (1993) Clozapine and N-desmethylclozapine are potent 5-HT1C receptor antagonists. Eur J Pharmacol 245:179-182.

Lee M A, Jayathilake K, Meltzer H Y (1999) A comparison of the effect of clozapine with typical neuroleptics on cognitive function in neuroleptic-responsive schizophrenia. Schizophr Res 37:1-11.

Leucht S, Wahlbeck K, Hamann J, Kissling W (2003) New generation antipsychotics versus low-potency conventional antipsychotics: a systematic review and meta-analysis. Lancet 361(9369):1581-1589.

Mauri M C, Volonteri L S, Dell'Osso B, Regispani F, Papa P, Baldi M, Bareggi S R (2003) Predictors of clinical outcome in schizophrenic patients responding to clozapine. J Clin Psychopharmacol 23(6):660-664.

Meltzer H Y, Matsubara S, Lee J C (1989) Classification of typical and atypical antipsychotic drugs on the basis of dopamine D-1, D-2 and serotonin2 pKi values. J Pharmacol Exp Ther 251:238-246.

Meltzer H Y, Alphs L, Green A I, Altamura A C, Anand R, Bertoldi A, Bourgeois M, Chouinard G, Zahur Islam M, Kane J, Krishnan R, Lindenmayer J P, Potkin S (2003) Clozapine treatment for suicidality in schizophrenia. Arch Gen Psychiatry 60:82-91.

Miller R J, Hiley C R (1974) Anti-muscarinic properties of neuroleptics and drug-induced Parkinsonism. Nature 248:596-597.

Mirza N R, Peters D, Sparks R G (2003) Xanomeline and the antipsychotic potential of muscarinic receptor subtype selective agonists. CNS Drug Rev 9(2):159-186.

Nordin C, Alme B, Bondesson U (1995) CSF and serum concentrations of clozapine and its demethyl metabolite: a pilot study. Psychopharmacology 122:104-107.

Olianas M C, Maullu C, Onali P (1999) Mixed agonist-antagonist properties of clozapine at different human cloned muscarinic receptor subtypes expressed in chinese hamster ovary cells. Neuropsychopharmacology 20(3):263-270.

Perry P J, Miller D D, Arndt S V, Cadoret R J (1991) Clozapine and norclozapine plasma concentrations and clinical response of treatment-refractory schizophrenic patients. Am J Psychiatry 148(2):231-135.

Pfeiffer C C, Jenney E H (1957) The inhibition of the conditioned response and the counteraction of schizophrenia by muscarinic stimulation of the brain. Ann NY Acad Sci 66:753-764.

Shannon B E, Bymaster F P, Calligaro D O, Greenwood B, Mitch C H, Sawyer B D, Ward J S, Wong D T, Olesen P H, Sheardown M J, Swedberg M D B, Suzdak P D, Sauerberg P (1994) Xanomeline: a novel muscarinic receptor agonist with functional selectivity for M1 receptors. J Pharmacol Exp Ther 269(1):271-281.

Shannon B E, Rasmussen K, Bymaster F P, Hart J C, Peters S C, Swedberg M D, Jeppesen L, Sheardown M J, Sauerberg P, Fink-Jensen A (2000) Xanomeline, an M(1)/M(4) preferring muscarinic cholinergic receptor agonist, produces antipsychotic-like activity in rats and mice. Schizophr Res 42:249-259.

Snyder S, Greenberg D, Yamamura H I (1974) Anti-schizophrenic drugs and brain cholinergic receptors. Affinity for muscarinic sites predicts extrapyramidal effects. Arch Gen Psychiatry 31:58-61.

Spalding T A, Trotter C, Skjaerbaek N, Messier T L, Currier E A, Burstein E S, Li D, Hacksell U, Brann M R (2002) Discovery of an ectopic activation site on the M(1) muscarinic receptor. Mol Pharmacol 61:1297-1302.

Spina E, Avenoso A, Facciola G, Salemi M, Scordo M G, Ancione M, Madia A G, Perucca E (2001) Relationship between plasma risperidone and 9-hydroxyrisperidone concentrations and clinical response in patients with schizophrenia. Psychopharmacology 153:238-243.

Sur C, Mallorga P J, Wittmann M, Jacobson M A, Pascarella D, Williams J B, Brandish P E, Pettibone D J, Scolnick E M, Conn, P J (2003) N-desmethylclozapine, an allosteric agonist at muscarinic 1 receptor, potentiates N-methyl-d-aspartate receptor activity. Proc Natl Acad Sci USA 100(23):13674-13679.

The Parkinson Study Group (1999) Low-dose clozapine for the treatment of drug-induced psychosis in Parkinson's Disease. N Engl J Med 340:757-763.

Weigmann H, Hartter S, Fischer V, Dahmen N, Hiemke C (1999) Distribution of clozapine and desmethylclozapine between blood and brain in rats. Eur. Neuropsychopharmacol 9:253-256.

Weiner D M, Levey A I, Brann M R (1990) Expression of muscarinic receptor acetylcholine and dopamine receptor mRNA's in rat basal ganglia. Proc Natl Acad Sci USA. 87:7050-7054.

Weiner D M, Burstein E S, Nash N, Croston G E, Currier E A, Vanover K E, Harvey S C, Donohue E, Hansen H C, Andersson C M, Spalding T A, Gibson D F C, Krebs-Thomson K, Powell, S B, Geyer M A, Hacksell U, Brann M R (2001) 5-hydroxytryptamine2a receptor inverse agonists as antipsychotics. J Pharmacol Exp Ther 299:268-276.

Weissman J T, Ma J, Essex A, Gao Y, Burstein E S (2003) G-protein-coupled receptor-mediated activation of rap GTPases: characterization of a novel Gi regulated pathway. Oncogene 23(1):241-249.

Wellendorph P, Goodman M W, Burstein E S, Nash N R, Brann M R, Weiner D M (2002) Molecular cloning and pharmacology of functionally distinct isoforms of the human histamine H3 receptor. Neuropharmacology 42:929-940.

Wong A H, Van Tol H H (2003) Schizophrenia: from phenomenology to neurobiology. Neurosci Biobehav Rev 27(3):269-306.

Young C D, Meltzer H Y, Deutch A Y (1998) Effects of desmethylclozapine on fos protein expression in the forebrain: in vivo biological activity of the clozapine metabolite. Neuropsychopharmacology 19:99-103.

Zorn S H, Jones S B, Ward K M, Liston D R (1994) Clozapine is a potent and selective muscarinic M4 receptor agonist. Eur J Pharmacol 269:R1-R2.

What is claimed is:

1. A method of treating psychosis of any origin, comprising:
administering a first dosage of N-desmethylclozapine to a patient exhibiting one or more symptoms of psychosis; and
subsequently administering a second dosage of N-desmethylclozapine to the patient, wherein the second dosage is higher than the first dosage.

2. The method of claim 1, comprising after administering the first dosage, identifying whether the patient would be sensitive to the second dosage.

3. The method of claim 1, wherein the first dosage is 25% of the second dosage.

4. The method of claim 1, wherein the first dosage is 50% of the second dosage.

5. The method of claim 1, wherein each dosage is administered BID.

6. The method of claim 1, wherein the patient has schizophrenia.

7. The method of claim 1, wherein the first dosage is a pre-conditioning dosage.

8. A method of treating psychosis of any origin, comprising administering a gradual dose escalation of N-desmethylclozapine to a patient exhibiting one or more symptoms of psychosis.
